(19) United States
(12) Patent Application Publication
(10) Pub. No.: US 2006/0160076 A1
(43) Pub. Date: Jul. 20, 2006

(54) Methods of treating and diagnosing diabetes with cx3cr1 modulators

(75) Inventors: Shonna Moodie, San Francisco, CA (US); Thomas A Gustafson, Danville, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: MEATABOLEX, INC., Hayward, CA (US)

(21) Appl. No.: 10/516,032

(22) PCT Filed: Jun. 10, 2003
(86) PCT No.: PCT/US03/18570

§ 371 Date: Nov. 24, 2004

Related U.S. Application Data
(60) Provisional application No. 60/387,699, filed on Jun. 10, 2002.

(51) Int. Cl. C12Q 1/68; C12Q 1/48; A61K 48/00; A61K 38/22

(52) U.S. Cl. 435/6; 435/15; 514/12; 514/44

(57) ABSTRACT

The present invention provides compositions and methods for diagnosing and treating diabetes and insulin resistance. In particular, the invention provides methods of identifying modulators of CX3CR1 and using those modulators to treat diabetes, as well as methods of diagnosing diabetes by measuring the levels of CX3CR1 or fractalkine in a patient.

### CROSS-REFERENCE TO RELATED APPLICATIONS

The present application claims benefit of priority to U.S. Provisional Patent Application No. 60/387,699, filed Jun. 10, 2002, which is incorporated by reference for all purposes.

### BACKGROUND OF THE INVENTION

Chemokines constitute a super family of small, inducible, secreted, proinflammatory cytokines involved in a variety of immune responses, acting primarily as chemoattractants and activators of specific types of leukocytes. Four classes of chemokines have been defined by the arrangement of the conserved cysteine (C) residues of the mature proteins: the CXC chemokines that have one amino acid residue separating the first two conserved cysteine residues; the CC chemokines in which the first two conserved cysteines residues are adjacent; the C chemokines that lack two (the first and third) of the four conserved cysteine residues; and the CX3C chemokines which have three intervening amino acids residues between the first two conserved cysteine residues. The C-C chemokines such as RANTES and MIP-1a, have been characterized as chemoattractants and activators of monocytes and lymphocytes. Members of the family of CC chemokines have been traditionally associated with chronic inflammatory disease such as asthma, arthritis and atherosclerosis, and implicated in regulating immune system homeostasis and autoimmunity. The C-X-C chemokines include a number of potent chemoattractants and activators of neutrophils, such as interleukin 8, platelet factor four and neutrophil-activating peptide-2. The CX3C chemokines include fractalkine (also known as SCYD1 and neurotactin in mice).

Chemokines mediate their activities by binding to target cell surface chemokine receptors that belong to the large family of G protein-coupled, seven transmembrane domain receptors (GPCRs). To date, four CXC chemokine receptors (CXCR-1 through 4), at least eight CC chemokine receptors (CCR-1 through 8) and one CX3C chemokine receptor (CX3CR) have been cloned and characterized. Chemokines induce their effects by binding to a GPCR, some of which are specific and interact with a single chemokine, whereas others—the so-called shared receptors—bind multiple ligands within, but not between, the CC and CXC branches. This super family of GPCRs all share within, but not between, the CC and CXC branches. This super family of GPCRs all share structural features that reflect a common mechanism of action of signal transduction. Conserved features include seven hydrophobic domains spanning the plasma membrane, which are connected by hydrophilic extracellular and intracellular loops.

The human CX3C chemokine receptor 1 (CX3CR1, also referred to as CXXXCR1, CMKBRL1 and V28) is expressed by a variety of different cells and tissues including peripheral blood leukocytes, neutrophils, monocytes and several solid organs, including brain and spleen (Raport et al., Gene 163:295-299 (1995); Combadiere et al. J. Biol. Chem. 273:23799-23804 (1998); Imai et al, Cell 91:521-530 (1997)). The murine counterpart, which has 83 percent amino acid identity to the human receptor gene, was identified by homology hybridization with the human receptor gene. The chemokine for the CX3CR1 receptor was recently identified as fractalkine (Hieshima et al., Cell 91:521 (1997)). CX3CR4 mediates both the adhesive and migratory functions of fractalkine.

Fractalkine, which like the receptor possesses the novel CX3C chemokine motif, has recently been identified. Human fractalkine n-mRNA expression is most abundant in the brain and heart, but is also present at lower levels in other tissues tested. Mouse fractalkine mRNA is also principally detected in brain and to a lesser extent in other tissues. Unlike other known chemokines, fractalkine is a type 1 membrane protein containing a chemokine domain at the amino terminus tethered to the plasma membrane on a long mucin-like stalk. Human fractalkine cDNA encodes a 397 amino acid residue membrane protein with a 24 amino acid residue predicted signal peptide, a 76 amino acid residue chemokine domain, a 24 amino acid residue stalk region containing 17 degenerate mucin-like repeats, a 19 amino acid residue transmembrane segment and a 37 amino acid residue cytoplasmic domain. The murine counterpart, neurotactin has overall 64% identity with higher identity observed within the conserved chemokine domain (77% identity). This mucin-chemokine hybrid type of protein can exist in two forms; either membrane-bound form or soluble secreted form. The membrane-bound form of fractalkine protein is markedly induced on primary endothelial cells by inflammatory cytokines, and it promotes strong adhesion of NK cells and CD8+ T cells. The soluble secreted form of fractalkine can be released, presumably by proteolysis at a membrane-proximal dibasic cleavage site, and has chemotactic activity for these leukocytes. The extracellular domain of fractalkine has been shown to be released into the supernatants of transfected cells by proteolysis at the dibasic cleavage site proximal to the membrane. Regulation of fractalkine cleavage is critical for maintaining the balance between the immobilized and soluble forms. Recent reports have identified tumor necrosis factor-α-converting enzyme (TACE) as being primarily responsible for generation of the inducible soluble form of fractalkine (Tsou et al., J. Biol. Chem., 276:44622-44626 (2001); Garton et al., J. Biol. Chem. 276:37993-38001 (2001)). Little or no inducible cleavage of fractalkine was evident in fibroblast derived from TACE −/− null mice (Tsou et al., J. Biol. Chem., 276:44622-44626 (2001)).

Fractalkine plays a central role in the trafficking of leukocytes in tissues with high blood flow. This function is attributed to its unique membrane bound structure, which enables it to form strong adhesive bonds with leukocytes expressing CX3CR1 receptor in this high shear environment (Fong et al., J. Biol. Chem. 275:3781-3786 (2000)). However fractalkine can also act as a typical cytokine in cell culture assays and in vivo since soluble forms of fractalkine are able to induce the migration of different types of T and natural killer cells through endothelial cells (Pan et al., Nature 387:611-617 (1997); Imai et al., Cell 91:521-530 (1997)). A recent study showed that fractalkine is cleaved from membranes of neurons in culture in response to an excitotoxic stimulus (Chapman et al., J. Neurosci. (Online), 20:RC87 (2000)). Fractalkine is also found to protect hippocampal neurons from the neurotoxicity induced by HIV-1 envelope protein gp120_{IIIB}. This effect is due to the fact that CX3CR1, like several other chemokine receptors, acts as a co-receptor for HIV virus entry into cells (Meucci et al., Proc. Natl. Acad. Sci. USA 97:8075-8080 (2000)).

Recently fractalkine has been proposed to have a role in rheumatoid arthritis. It has been shown to mediate angiogenesis of human dermal microvascular endothelial cells. Elevated levels of fractalkine were detected in synovial fluid from patients with osteoarthritis or other forms of arthritis (see, e.g., Ruth et al., Arthritis Rheum. 44:1568-1581 (2001); Volin et al., Am. J. Pathol. 159:1521-1530 (2001); WO 01/60406). Immunodepletion of fractalkine from rheumatoid arthritis synovial tissue homogenates inhibited the ability of the synovial tissue to induce angiogenesis in vivo.

Some signal transduction pathways that are induced by the receptor-ligand interaction for this family of chemokine and its receptor have been described. GPCRs are classically coupled to heterotrimeric G-proteins, of which at least 17 alpha (α), 4 beta (β), and 7 gamma (γ) subunits have been identified. Upon ligand binding to the GPCR, the G protein heterodimer dissociates into an active GTP bound α subunit and a βγ subunit. This triggers a series of downstream signals. The activated Gα subunit leads to modulation of the activity of ion channels, adenylate cyclase, phospholipases and phosphodiesterases. This leads to changes in the intracellular levels of a variety of intracellular signalling mediators including inositol trisphosphate, diacylglycerol, cyclic AMP and calcium. The βγ subunits activate the γ subunit of p110 catalytic subunit of PI3-kinase leading to the activation of the Ras→MAPK signalling pathway. Activation of CX3CR1 has been demonstrated to activate some of these downstream events. CHO cells stably overexpressing CX3CR1 treated with fractalkine results in an influx of extracellular calcium and subsequent activation of the MAPK pathway in a P13-kinase dependent manner (Kansra et al., J. Biol. Chem., 276:31831-31838 (2001)). Both calcium influx and MAPK activation could be blocked by pretreatment with the PI3-kinase inhibitors, wortmannin or LY294002. Receptor activation by soluble fractalkine in hippocampal neurons induces activation of the protein kinase AKT/PKB and the nuclear translocation of NF-κB to the nucleus (Meucci et al., Proc. Natl. Acad. Sci. USA, 97:8075-8080 (2000)). In addition, fractalkine was shown to activate two non-receptor tyrosine kinases, p60 src and p72 syk (Cambien et al., Blood, 97:2031-2037 (2001)).

Recent studies have shown an association with CX3CR1 in coronary vascular endothelial dysfunction and atherosclerosis. The study shows that a CX3CR1 polymorphism V249I affects receptor expression and function. An association was observed between the presence of the CX3CR1 I249 allele and reduced prevalence of coronary artery disease (McDermott et al., Circ. Res., 89:401-407 (2001); Moatti D., Blood 97:1925-1928 (2001)). The V249 I as well as a second polymorphism T280M are also found to be associated with an increased progression of AIDS (Faure et al., Science 287:2274-2277 (2000)).

Diabetes mellitus can be divided into two clinical syndromes, Type 1 and Type 2 diabetes mellitus. Type 1, or insulin-dependent diabetes mellitus (IDDM), is a chronic autoimmune disease characterized by the extensive loss of beta cells in the pancreatic Islets of Langerhans, which produce insulin. As these cells are progressively destroyed, the amount of secreted insulin decreases, eventually leading to hyperglycemia (abnormally high level of glucose in the blood) when the amount of secreted insulin drops below the level required for euglycemia (normal blood glucose level). Although the exact trigger for this immune response is not known, patients with IDDM have high levels of antibodies against proteins expressed in pancreatic beta cells. However, not all patients with high levels of these antibodies develop IDDM.

Type 2 diabetes (also referred to as non-insulin dependent diabetes mellitus (NIDDM)) develops when muscle, fat and liver cells fail to respond normally to insulin. This failure to respond (called insulin resistance) may be due to reduced numbers of insulin receptors on these cells, or a dysfunction of signaling pathways within the cells, or both. The beta cells initially compensate for this insulin resistance by increasing insulin output. Over time, these cells become unable to produce enough insulin to maintain normal glucose levels, indicating progression to Type 2 diabetes.

Type 2 diabetes is brought on by a combination of genetic and acquired risk factors—including a high-fat diet, lack of exercise, and aging. Worldwide, Type 2 diabetes has become an epidemic, driven by increases in obesity and a sedentary lifestyle, widespread adoption of western dietary habits, and the general aging of the population in many countries. In 1985, an estimated 30 million people worldwide had diabetes—by 2000, this figure had increased 5-fold, to an estimated 154 million people. The number of people with diabetes is expected to double between now and 2025, to about 300 millions.

Type 2 diabetes is a complex disease characterized by defects in glucose and lipid metabolism. Typically there are perturbations in many metabolic parameters including increases in fasting plasma glucose levels, free fatty acid levels and triglyceride levels, as well as a decrease in the ratio of HDL/LDL. As discussed above, one of the principal underlying causes of diabetes is thought to be an increase in insulin resistance in peripheral tissues, principally muscle and fat. The present invention addresses this and other problems.

### BRIEF SUMMARY OF THE INVENTION

The present invention provides methods of identifying an agent for treating a patient having diabetes or a predisposition for diabetes. In some embodiments, the methods comprise the steps of: (i) contacting a solution comprising an CX3CR1 polypeptide or ligand-binding fragment thereof with the agent, wherein the CX3CR1 polypeptide or ligand-binding fragment thereof is encoded by a nucleic acid that hybridizes under stringent conditions to a nucleic acid encoding a polypeptide having an amino acid sequence selected from the group consisting of SEQ ID NO:8, SEQ ID NO:10 and SEQ ID NO:12; and (ii) selecting an agent that increases the expression or activity of the CX3CR1 polypeptide or ligand-binding fragment thereof, thereby identifying an agent for treating a patient having diabetes or a predisposition for diabetes.

In some embodiments, the methods further comprise selecting an agent that modulates insulin sensitivity. In some embodiments, step (ii) comprises selecting an agent that increases expression of the CX3CR1 polypeptide. In some embodiments, step (ii) comprises selecting an agent that increases the activity of the CX3CR1 polypeptide. In some embodiments, the methods comprise contacting a cell expressing a CX3CR1 polypeptide or ligand-binding fragment thereof. In some embodiments, the methods comprise detecting AKT/PKB phosphorylation. In some embodiments, the cell is not treated with insulin. In some embodiments, the cell is treated with insulin. In some embodiments, the methods comprise detecting kinase activity of AKT/PKB. In some embodiments, the methods comprise detecting p42/p44 MAP kinase phosphorylation. In some embodiments, the methods comprise detecting p42/p44 MAP kinase activity. In some embodiments, the methods comprise detecting phosphorylation of SAPK/JNK1 or p38/SAPK2. In some embodiments, the methods comprise detecting activity of SAPK/JNK1 or p38/SAPK2. In some embodiments, the above-described kinase activities are detected in vitro. In some embodiments, the methods comprise detecting calcium flux in a cell. In some embodiments, the contacting step is performed in vitro.

In some embodiments, the CX3CR1 polypeptide or ligand-binding fragment thereof is expressed in a cell and the cell is contacted with the agent. In some embodiments, the methods comprise administering the agent to an animal having diabetes and testing the animal for decreased blood glucose levels compared to blood glucose levels before administration of the agent.

In some embodiments, the methods comprise administering the agent to an animal exhibiting insulin resistance and testing the animal for decreased insulin levels compared to insulin levels before administration of the agent. In some embodiments, the methods further comprise the steps of contacting a cell expressing a CX3CR1 polypeptide or ligand-binding fragment thereof with the agent and testing the cell for modulated insulin sensitivity. In some embodiments, insulin sensitivity is measured as a function of GLUT4 translocation or glucose uptake.

In some embodiments, the amino acid sequence comprises SEQ ID NO:8. In some embodiments, the amino acid sequence comprises SEQ ID NO:10. In some embodiments, the amino acid sequence comprises SEQ ID NO:12.

The present invention also provides methods of treating a prediabetic or diabetic animal. In some embodiments, the methods comprise administering a therapeutically effective amount of an agent that increases CX3CR1 activity or expression. In some embodiments, the methods comprise administering a therapeutically effective amount of an agent identified by the methods described above. In some embodiments, the animal is a human. In some embodiments, the animal is prediabetic. In some embodiments, the animal is diabetic.

In some embodiments, methods comprise administering a therapeutically effective amount of a polypeptide comprising an amino acid sequence at least 70% identical to SEQ ID NO:13. In some embodiments, the polypeptide sequence comprises SEQ ID NO:2. In some embodiments, the amino acid sequence comprises SEQ ID NO:13.

The present invention also provides methods of introducing an expression cassette into a cell. In some embodiments, the methods comprise introducing into the cell an expression cassette comprising a promoter operably linked to a polynucleotide encoding a polypeptide comprising an amino acid sequence at least 70% identical to SEQ ID NO:13. In some embodiments, the polypeptide comprises SEQ ID NO:13. In some embodiments, the polypeptide comprises SEQ ID NO:2. In some embodiments, the cell is selected from the group consisting of adipocytes, skeletal muscle, liver and blood cells. In some embodiments, the cell is introduced into a patient. In some embodiments, the patient is diabetic. In some embodiments, the patient is prediabetic. In some embodiments, the cell is from the patient. In some embodiments, the expression cassette is introduced into the cell in a viral vector.

The present invention also provides methods of diagnosing Type 2 diabetes or prediabetic individuals. In some embodiments, the methods comprise detecting in a sample from the individual the level of a CX3CR1 polypeptide or the level of a polynucleotide encoding a CX3CR1 polypeptide, wherein the CX3CR1 polypeptide is encoded by a nucleic acid that hybridizes under stringent conditions to a nucleic acid encoding a polypeptide having an amino acid sequence selected from the group consisting of SEQ ID NO:8, SEQ ID NO:10, and SEQ ID NO:12, wherein an increased level of the polypeptide or polynucleotide in the sample compared to a level of the polypeptide or polynucleotide in either a lean person or a previous sample from the individual indicates that the individual is diabetic or prediabetic.

In some embodiments, the detecting step comprises contacting the sample with an antibody that specifically binds to the CX3CR1 polypeptide. In some embodiments, the amino acid sequence comprises SEQ ID NO:8. In some embodiments, the amino acid sequence comprises SEQ ID NO:10. In some embodiments, the amino acid sequence comprises SEQ ID NO:12.

In some embodiments, the detecting step comprises quantifying mRNA encoding the CX3CR1 polypeptide. In some embodiments, the mRNA is reverse transcribed and amplified in a polymerase chain reaction. In some embodiments, the sample is a tissue sample.

In some embodiments, the methods comprise detecting in a biological sample from the patient the level of fractalkine, wherein an increased level of the fractalkine in the sample compared to a level of fractalkine in either a lean individual or a previous sample from the patient indicates that the patient is diabetic or prediabetic; and wherein the fractalkine polypeptide is encoded by a nucleic acid that hybridizes under stringent conditions to a nucleic acid encoding a polypeptide having an amino acid sequence selected from the group consisting of SEQ ID NO:2, SEQ ID NO:4, SEQ ID NO:6 and SEQ ID NO:13.

In some embodiments, the detecting step comprises contacting the sample with an antibody that specifically binds to fractalkine. In some embodiments, the sample is selected from the group consisting of a tissue sample, blood sample, saliva sample, and urine sample. In some embodiments, the polypeptide comprises SEQ ID NO:13. In some embodiments, the polypeptide comprises SEQ ID NO:2.

### Definitions

“Insulin sensitivity” refers to the ability of a cell or tissue to respond to insulin. Responses include, e.g., glucose uptake of a cell or tissue in response to insulin stimulation. Sensitivity can be determined at an organismal, tissue or cellular level. For example, blood or urine glucose levels following a glucose tolerance test are indicative of insulin sensitivity. Other methods of measuring insulin sensitivity include, e.g., measuring glucose uptake (see, e.g., Garcia de Herreros, A., and Birnbaum, M. J. J. Biol. Chem. 264, 19994-19999 (1989); Klip, A., Li, G., and Logan, W. J. Am. J. Physiol. 247, E291-296 (1984)), measuring the glucose infusion rate (GINF) into tissue such as the skeletal muscle (see, e.g., Ludvik et al., J Clin. Invest. 100:2354 (1997); Frias et al., Diabetes Care 23:64, (2000)) and measuring sensitivity of GLUT4 translocation (e.g., as described herein) in response to insulin.

“CX3CR1 activity” refers to the ability of a CX3CR1 polypeptide to bind ligands such as fractalkine and/or to transduce a signal Activity can be measured by, e.g., simple competitive binding assays to determine ligand binding, by measuring physiological (e.g., calcium or inositol phosphate changes) or biochemical responses to CX3CR1 in a cell (e.g., activation or phosphorylation of downstream proteins such as AKT/PKB, MAPK, SAPK/JNK, p38/SAPK2 and the like) as described herein or known to those of skill in the art.

“Ligand binding fragment” refers to an amino acid sequence (such as CX3CR1) capable of binding to a chemokine ligand such as fractalkine.

“Predisposition for diabetes” occurs in a person when the person is at high risk for developing diabetes. A number of risk factors are known to those of skill in the art and include: genetic factors (e.g., carrying alleles that result in a higher occurrence of diabetes than in the average population or having parents or siblings with diabetes); overweight (e.g., body mass index (BMI) greater or equal to 25 kg/m²); habitual physical inactivity, race/ethnicity (e.g., African-American, Hispanic-American, Native Americans, Asian-Americans, Pacific Islanders); previously identified impaired fasting glucose or impaired glucose tolerance, hypertension (e.g., greater or equal to 140/90 mmHg in adults); HDL cholesterol greater or equal to 35 mg/dl; triglyceride levels greater or equal to 250 mg/dl; a history of gestational diabetes or delivery of a baby over nine pounds; and/or polycystic ovary syndrome. See, e.g., “Report of the Expert Committee on the Diagnosis and Classification of Diabetes Mellitus” and “Screening for Diabetes” Diabetes Care 25(1): S5-S24 (2002).

A “lean individual,” when used to compare with a sample from a patient, refers to an adult with a fasting blood glucose level less than 110 mg/dl or a 2 hour PG reading of 140 mg/dl. “Fasting” refers to no caloric intake for at least 8 hours. A “2 hour PG” refers to the level of blood glucose after challenging a patient to a glucose load containing the equivalent of 75 g anhydrous glucose dissolved in water. The overall test is generally referred to as an oral glucose tolerance test (OGTT). See, e.g., Diabetes Care, Supplement 2002, American Diabetes Association: Clinical Practice Recommendations 2002. The level of a polypeptide in a lean individual can be a reading from a single individual, but is typically a statistically relevant average from a group of lean individuals. The level of a polypeptide in a lean individual can be represented by a value, for example in a computer program.

A “pre-diabetic individual,” when used to compare with a sample from a patient, refers to an adult with a fasting blood glucose level greater than 110 mg/dl but less than 126 mg/dl or a 2 hour PG reading of greater than 140 mg/dl but less than 200 mg/dl. A “diabetic individual,” when used to compare with a sample from a patient, refers to an adult with a fasting blood glucose level greater than 126 mg/dl or a 2 hour PG reading of greater than 200 mg/dl.

A “CX3CR1 nucleic acid” or “CX3CR1 polynucleotide” of the invention is a subsequence or full-length polynucleotide sequence of a gene that encodes an CX3CR1 polypeptide. Exemplary CX3CR1 nucleic acids of the invention include sequences substantially identical to CX3CR1 (see, e.g., SEQ ID NOs: 7, 9 and 11). Exemplary CX3CR1 polynucleotides encode, e.g., SEQ ID NOs:8, 10 and 12. The nucleotide sequence encoding human CX3C chemokine receptor 1 (CX3CR1) is deposited in Genbank under the Accession number of U20350 has an open reading frame beginning at position 88 and ending with a stop codon at position 1155. See, SEQ ID NO:7. The nucleotide sequence encoding mouse CX3C chemokine receptor 1 (CX3CR1) is deposited in Genbank under the Accession number of AF074912 and has an open reading frame beginning at position 220 and ending with a stop codon at position 1284. See SEQ ID NO:9. The nucleotide sequence encoding rat CX3C chemokine receptor 1 (CX3CR1) is deposited in Genbank under the Accession number of U04808 and has an open reading frame beginning at position 68 and ending with a stop codon at position 1132. See SEQ ID NO:11.

“CX3CR1 polypeptide” or “CX3CR1” refers to a polypeptide, or fragment thereof, that is substantially identical to a polypeptide encoded by CX3CR1 (e.g., SEQ ID NO:7, SEQ ID NO:9, and SEQ ID NO:11. CX3CR1 is a G-protein coupled receptor (GPCR) GPCR structure is known to those of skill in the art and comprises at least three domains: an N-terminal ligand binding domain, and transmembrane domain (seven transmembrane regions) and a C-terminal signal transduction domain. CX3CR1 polypeptides have the ability to bind the chemokine fractalkine and typically can transduce a signal as a result of that binding.

“Fractalkine” refers to a chemokine capable of binding CX3CR1. See, e.g., Fong et al. J. Biol. Chem. 275:3781-3786 (2000); Imai et al., Cell 91(4):521-30 (1997). Exemplary fractalkine polypeptides include, e.g., SEQ ID NOs:2, 4, and 6. A nucleotide sequence encoding human-fractalkine is deposited in Genbank under the Accession number NM_{—}002996 and has an open reading frame beginning at position 80 and ending with a stop codon at position 1273. See, also, SEQ ID NO:1. Nucleotides 80-151 encode the signal peptide, nucleotides 152-1270 encode the mature peptide, nucleotides 152-379 encode the chemokine module, nucleotides 380-1102 encode the glycosylation stalk, nucleotides 1103-1159 encode the transmembrane helix, and nucleotides 1160-1270 encode the intracellular domain. The nucleotide sequence encoding mouse fractalkine deposited in Genbank under the Accession number AF071549, having an open reading frame beginning at position 77 and ending with a stop codon at position 1264. The nucleotide sequence encoding rat fractalkine deposited in Genbank under the Accession number AF030358, having an open reading frame beginning at position 21 and ending with the stop codon at position 1202. See, SEQ ID NO:5. The chemokine domain of human fractalkine is displayed in SEQ ID NO:13 (amino acid) and SEQ ID NO:14 (nucleotide). A structure/function mutational analysis has been performed and reveals residues the effect fractalkine function. See, e.g., Harrison, et al., J. Biol. Chem. 276(24):21632-21641 (2001).

Chemokines exhibit a conserved structure, which features a core globular β barrel established by three anti-parallel β-strands. See, e.g., Clark-Lewis et al., J. Leukoc. Biol. 5745 (522):703-711 (1995); Lusti-Narasimhan et al., J. Biol. Chem. 270:2716-2721 (1995); Schwarz & Wells, Curr. Opin. In Chem. Biol. 3:407-417 (1999); Fernandez & Lolis, Annu. Rev. Pharmacol. Toxicol. 42:469-499 (2002). This core is flanked by a highly basic C-terminal α-helix and a short, relatively disordered N-terminal segment. The N-terminal segment contains much, and sometimes all, of the structural information required for receptor specificity. The core structure of chemokines is maintained in part by the disulfide bonds between the cysteine residues that are positionally conserved. Chemokines share the same three-dimensional fold despite a sequence identity of less than 20%.

An “agonist of CX3CR1” refers to an agent that binds to CX3CR1, stimulates, increases, activates, facilitates, enhances activation, sensitizes or up regulates the activity or expression of CX3CR1.

An “antagonist of CX3CR1” refers to an agent that binds to, partially or totally blocks stimulation, decreases, prevents, delays activation, inactivates, desensitizes, or down regulates the activity or expression of CX3CR1.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The terms “peptidomimetic” and “mimetic” refer to a synthetic chemical compound that has substantially the same structural and functional characteristics of the CX3CR1 antagonists or agonists of the invention. Peptide analogs are commonly used in the pharmaceutical industry as non-peptide drugs with properties analogous to those of the template peptide. These types of non-peptide compound are termed “peptide mimetics” or “peptidomimetics” (Fauchere, J. Adv. Drug Res. 15:29 (1986); Veber and Freidinger TINS p. 392 (1985); and Evans et al. J. Med. Chem. 30:1229 (1987), which are incorporated herein by reference). Peptide mimetics that are structurally similar to therapeutically useful peptides may be used to produce an equivalent or enhanced therapeutic or prophylactic effect. Generally, peptidomimetics are structurally similar to a paradigm polypeptide (i.e., a polypeptide that has a biological or pharmacological activity), such as an CX3CR1 polypeptide agonist, but have one or more peptide linkages optionally replaced by a linkage selected from the group consisting of, e.g., —CH2NH—, —CH2S—, —CH2-CH2-, —CH═CH— (cis and trans), —COCH2-, —CH(OH)CH2-, and —CH2SO—. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. For example, a mimetic composition is within the scope of the invention if it is capable of carrying out the binding or other activities of a CX3CR1 agonist or antagonist.

The term “gene” means the segment of DNA involved in producing a polypeptide chain; it includes regions preceding and following the coding region (leader and trailer) as well as intervening sequences (introns) between individual coding segments (exons).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). The term nucleic acid is used interchangeably with gene, cDNA, and mRNA encoded by a gene.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

The term “similarity,” or percent “similarity,” in the context of two or more polypeptide sequences, refer to two or more sequences or subsequences that have a specified percentage of amino acid residues that are either the same or similar as defined in the 8 conservative amino acid substitutions defined above (i.e., 60%, optionally 65%, 70%, 75%, 80%, 85%, 90%, 95%, 98% or 99% similar over a specified region or, when not specified, over the entire sequence), when compared and aligned for maximum correspondence over a comparison window, or designated region as measured using one of the following sequence comparison algorithms or by manual alignment and visual inspection. Such sequences are then said to be “substantially similar.” Optionally, this identity exists over a region that is at least about 50 amino acids in length, or more preferably over a region that is at least about 100, 200, 300, 400, 500 or 1000 or more amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al. (1984) Nuc. Acids Res. 12:387-395).

Another example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

The phrase “a nucleic acid sequence encoding” refers to a nucleic acid which contains sequence information for a structural RNA such as rRNA, a tRNA, or the primary amino acid sequence of a specific protein or peptide, or a binding site for a transacting regulatory agent. This phrase specifically encompasses degenerate codons (i.e., different codons which encode a single amino acid) of the native sequence or sequences that may be introduced to conform with codon preference in a specific host cell.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under-expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “specifically (or selectively) binds to an antibody” or “specifically (or selectively) immunoreactive with”, when referring to a protein or peptide, refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. For example, antibodies raised against a protein having an amino acid sequence encoded by any of the polynucleotides of the invention can be selected to obtain antibodies specifically immunoreactive with that protein and not with other proteins, except for polymorphic variants. A variety of immunoassay formats may be used to select antibodies specifically immunoreactive with a particular protein. For example, solid-phase ELISA immunoassays, Western blots, or immunohistochemistry are routinely used to select monoclonal antibodies specifically immunoreactive with a protein. See, Harlow and Lane Antibodies, A Laboratory Manual, Cold Spring Harbor Publications, NY (1988) for a description of immunoassay formats and conditions that can be used to determine specific immunoreactivity. Typically, a specific or selective reaction will be at least twice the background signal or noise and more typically more than 10 to 100 times background.

“Inhibitors,” “activators,” and “modulators” of CX3CR1 expression or of CX3CR1 activity are used to refer to inhibitory, activating, or modulating molecules, respectively, identified using in vitro and in vivo assays for CX3CR1 expression or CX3CR1 activity, e.g., ligands, agonists, antagonists, and their homologs and mimetics. The term “modulator” includes inhibitors and activators. Inhibitors are agents that, e.g., inhibit expression of CX3CR1 or bind to, partially or totally block stimulation, decrease, prevent, delay activation, inactivate, desensitize, or down regulate the activity of CX3CR1, e.g., antagonists. Activators are agents that, e.g., induce or activate the expression of a CX3CR1 or bind to, stimulate, increase, open, activate, facilitate, or enhance activation, sensitize or up regulate the activity of CX3CR1, e.g., agonists. Modulators include naturally occurring and synthetic ligands, antagonists, agonists, small chemical molecules and the like. Such assays for inhibitors and activators include, e.g., applying putative modulator compounds to cells expressing CX3CR1 and then determining the functional effects on CX3CR1 activity, as described above. Samples or assays comprising CX3CR1 that are treated with a potential activator, inhibitor, or modulator are compared to control samples without the inhibitor, activator, or modulator to examine the extent of effect. Control samples (untreated with modulators) are assigned a relative CX3CR1 activity value of 100%. Inhibition of CX3CR1 is achieved when the CX3CR1 activity value relative to the control is about 80%, optionally 50% or 25, 10%, 5% or 1%. Activation of CX3CR1 is achieved when the CX3CR1 activity value relative to the control is 110%, optionally 150%, optionally 200, 300%, 400%, 500%, or 1000-3000% or more higher.

### BRIEF DESCRIPTION OF THE DRAWINGS

FIGS. 1 through 3 are bar graphs illustrating the expression levels of fractalkine and CX3CR1 mRNA in the skeletal muscle of humans.

FIGS. 4A and 4B are bar graphs illustrating the expression levels of mouse neurotactin (fractalkine) and CX3CR1 mRNA in the skeletal muscle and adipose from diet-induced insulin resistant mice. The Students t-test was used to compare groups. Values of p≦0.05 were considered significant.

FIG. 5A illustrates a western blot bound with anti-phosphoserine 473-AKT/PKB rabbit polyclonal antibodies.

FIG. 5B is a bar garph illustrating the effect of increasing concentrations of fractalkine on insulin-stimulated glucose uptake in adipocytes.

### DETAILED DESCRIPTION OF THE INVENTION

I. Introduction

The present application demonstrates that, surprisingly, elevated levels of CX3CR1 mRNA occur in skelatal muscle from both insulin resistant obese, non-diabetic individuals and type 2 diabetic individuals. In addition, levels of fractalkine (the chemokine ligand of CX3CR1) are also elevated in human skeletal muscle from type 2 diabetics. A rodent model of diet induced obesity and insulin resistance also displayed elevated expression levels of CX3CR1 and neurotactin in skeletal muscle. Moreover, pretreatment of adipocytes with the chemokine domain of recombinant fractalkine increases insulin-stimulated glucose uptake. Thus, without intending to limit the invention to a particular mechanism of action, it is believed that upregulation of CX3CR1 activity is beneficial in treating diabetic, pre-diabetic or obese insulin resistant, non-diabetic patients. Furthermore, elevated levels of CX3CR1 or fractalkine are indicative of insulin resistance. Thus, the detection of either CX3CR1 or fractalkine is useful for diagnosis of diabetes and insulin resistance.

This invention provides methods of using CX3CR1 sequences and modulators of CX3CR1 to diagnose and treat diabetes and related metabolic diseases. The present method also provides methods of identifying modulators of CX3CR1 expression and activity. Such modulators are useful for treating Type 2 diabetes as well as the pathological aspects of diabetes.

II. General Recombinant Nucleic Acid Methods for Use with the Invention

In numerous embodiments of the present invention, nucleic acids encoding a CX3CR1 or fractalkine of interest will be isolated and cloned using recombinant methods. Such embodiments are used, e.g., to isolate CX3CR1 polynucleotides (e.g., SEQ ID NO: 7, SEQ ID NO: 9, and SEQ ID NO:11) and fractalkine polynucleotides (e.g., SEQ ID NO:1, SEQ ID NO:3 and SEQ ID NO:5) for protein expression or during the generation of variants, derivatives, expression cassettes, or other sequences derived from an CX3CR1 polypeptide (e.g., SEQ ID NO:8, SEQ ID NO:10, and SEQ ID NO:12) or a fractalkine polypeptide (e.g., SEQ ID NO:2, SEQ ID NO:4, and SEQ ID NO:6), to monitor gene expression, for the isolation or detection of CX3CR1 or fractalkine sequences in different species, for diagnostic purposes in a patient, e.g., to detect mutations in CX3CR1 or fractalkine or to detect expression levels of nucleic acids or polypeptides. In some embodiments, the sequences encoding the polypeptides of the invention are operably linked to a heterologous promoter. In one embodiment, the nucleic acids of the invention are from any mammal, including, in particular, e.g., a human, a mouse, a rat, etc.

A. General Recombinant Nucleic Acid Methods

This invention relies on routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994)).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981).

B. Cloning Methods for the Isolation of Nucleotide Sequences Encoding Desired Proteins

In general, the nucleic acids encoding the subject proteins are cloned from DNA sequence libraries that are made to encode cDNA or genomic DNA. The particular sequences can be located by hybridizing with an oligonucleotide probe, the sequent of which can be derived from the sequences disclosed herein, which provide a reference for PCR primers and defines suitable regions for isolating CX3CR1 and fractalkine specific probes. Alternatively, where the sequence is cloned into an expression library, the expressed recombinant protein can be detected immunologically with antisera or purified antibodies made against a polypeptide of interest, including those disclosed herein.

Methods for making and screening genomic and cDNA libraries are well known to those of skill in the art (see, e.g., Gubler and Hoffman Gene 25:263-269 (1983); Benton and Davis Science, 196:180-182 (1977); and Sambrook, supra). Heart and brain cells are an example of suitable cells to isolate fractalkine RNA. CX3CR1 RNA can be isolated from peripheral blood leukocytes, neutrophils, and monocytes, as well as brain and spleen, for example.

Briefly, to make the cDNA library, one should choose a source that is rich in mRNA. The mRNA can then be made into cDNA, ligated into a recombinant vector, and transfected into a recombinant host for propagation, screening and cloning. For a genomic library, the DNA is extracted from a suitable tissue and either mechanically sheared or enzymatically digested to yield fragments of preferably about 5-100 kb. The fragments are then separated by gradient centrifugation from undesired sizes and are constructed in bacteriophage lambda vectors. These vectors and phage are packaged in vitro, and the recombinant phages are analyzed by plaque hybridization. Colony hybridization is carried out as generally described in Grunstein et al., Proc. Natl. Acad. Sci. USA., 72:3961-3965 (1975).

An alternative method combines the use of synthetic oligonucleotide primers with polymerase extension on an mRNA or DNA template. Suitable primers can be designed from specific CX3CR1 or fractalkine sequences disclosed herein. This polymerase chain reaction (PCR) method amplifies the nucleic acids encoding the protein of interest directly from mRNA, cDNA, genomic libraries or cDNA libraries. Restriction endonuclease sites can be incorporated into the primers. Polymerase chain reaction or other in vitro amplification methods may also be useful, for example, to clone nucleic acids encoding specific proteins and express said proteins, to synthesize nucleic acids that will be used as probes for detecting the presence of mRNA encoding a polypeptide of the invention in physiological samples, for nucleic acid sequencing, or for other purposes (see, U.S. Pat. Nos. 4,683,195 and 4,683,202). Genes amplified by a PCR reaction can be purified from agarose gels and cloned into an appropriate vector.

Appropriate primers and probes for identifying the genes encoding a polypeptide of the invention from mammalian tissues can be derived from the sequences provided herein. For a general overview of PCR, see, Innis et al. PCR Protocols: A Guide to Methods and Applications, Academic Press, San Diego (1990).

Synthetic oligonucleotides can be used to construct genes. This is done using a series of overlapping oligonucleotides, usually 40-120 bp in length, representing both the sense and anti-sense strands of the gene. These DNA fragments are then annealed, ligated and cloned.

A polynucleotide encoding a CX3CR1 or fractalkine polypeptide of the invention can be cloned using intermediate vectors before transformation into mammalian cells for expression. These intermediate vectors are typically prokaryote vectors or shuttle vectors. The proteins can be expressed in either prokaryotes or eukaryotes, using standard methods well known to those of skill in the art.

III. Purification of Proteins of the Invention

Either naturally occurring or recombinant CX3CR1 or fractalkine can be purified for use in functional assays. Naturally occurring CX3CR1 can be purified, e.g., from leukocytes, neutrophils, monocytes, brain, spleen or any other source of a CX3CR1 ortholog. Similarly, naturally occurring fractalkine can be purified, e.g., from heart or brain tissue or any other source of a fractalkine ortholog. Recombinant polypeptides can be purified from any suitable expression system.

The polypeptides of the invention may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Sambrook et al., supra).

A number of procedures can be employed when recombinant polypeptides are being purified. For example, proteins having established molecular adhesion properties can be reversibly fused to CX3CR1 or fractalkine. With the appropriate ligand, either protein can be selectively adsorbed to a purification column and then freed from the column in a relatively pure form. The fused protein may be then removed by enzymatic activity. Finally polypeptides can be purified using immunoaffinity columns.

A. Purification of Proteins from Recombinant Bacteria

When recombinant proteins are expressed by the transformed bacteria in large amounts, typically after promoter induction, although expression can be constitutive, the proteins may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells typically, but not limited to, by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook et al., both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, are inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques.

Alternatively, it is possible to purify proteins from bacteria periplasm. Where the protein is exported into the periplasm of the bacteria, the periplasmic fraction of the bacteria can be isolated by cold osmotic shock in addition to other methods known to those of skill in the art (see, Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

B. Purification of Proteins from Insect Cells

Proteins can also be purified from eukaryotic gene expression systems as described in, e.g., Fernandez and Hoeffler, Gene Expression Systems (1999). In some embodiments, baculovirus expression systems are used to isolate proteins of the invention. Recombinant baculoviruses are generally generated by replacing the polyhedrin coding sequence of a baculovirus with a gene to be expressed (e.g., a CX3CR1 or fractalkine polynucleotide). Viruses lacking the polyhedrin gene have a unique plaque morphology making them easy to recognize. In some embodiments, a recombinant baculovirus is generated by first cloning a polynucleotide of interest into a transfer vector (e.g., a pUC based vector) such that the polynucleotide is operably linked to a polyhedrin promoter. The transfer vector is transfected with wildtype DNA into an insect cell (e.g., Sf9, Sf21 or BT1-TN-5B14 cells), resulting in homologous recombination and replacement of the polyhedrin gene in the wildtype viral DNA with the polynucleotide of interest. Virus can then be generated and plaque purified. Protein expression results upon viral infection of insect cells. Expressed proteins can be harvested from cell supernatant if secreted, or from cell lysates if intracellular. See, e.g., Ausubel et al. and Fernandez and Hoeffler, supra.

C. Standard Protein Separation Techniques for Purifying Proteins

1. Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

2. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of the protein of interest. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

3. Column Chromatography

The proteins of interest can also be separated from other proteins on the basis of their size, net surface charge, hydrophobicity and affinity for ligands. In addition, antibodies raised against proteins can be conjugated to column matrices and the proteins immunopurified. All of these methods are well known in the art.

Immunoaffinity chromatography using antibodies raised to a variety of affinity tags such as hemagglutinin (HA), FLAG, Xpress, Myc, hexahistidine (His), glutathione S transferase (GST) and the like can be used to purify polypeptides. The His tag will also act as a chelating agent for certain metals (e.g., Ni) and thus the metals can also be used to purify His-containing polypeptides. After purification, the tag is optionally removed by specific proteolytic cleavage.

It will be apparent to one of skill that chromatographic techniques can be performed at any scale and using equipment from many different manufacturers (e.g., Pharmacia Biotech).

IV. Detection of Polynucleotides of the Invention

Those of skill in the art will recognize that detection of expression of CX3CR1 or fractalkine polynucleotides has many uses. For example, as discussed herein, detection of CX3CR1 or fractalkine levels in a patient is useful for diagnosing diabetes or a predisposition for at least some of the pathological effects of diabetes. Moreover, detection of gene expression is useful to identify modulators of CX3CR1 or fractalkine expression.

A variety of methods of specific DNA and RNA measurement that use nucleic acid hybridization techniques are known to those of skill in the art (see, Sambrook, supra). Some methods involve an electrophoretic separation (e.g., Southern blot for detecting DNA, and Northern blot for detecting RNA), but measurement of DNA and RNA can also be carried out in the absence of electrophoretic separation (e.g., by dot blot). Southern blot of genomic DNA (e.g., from a human) can be used for screening for restriction fragment length polymorphism (RFLP) to detect the presence of a genetic disorder affecting a CX3CR1 or fractalkine polypeptide of the invention.

The selection of a nucleic acid hybridization format is not critical. A variety of nucleic acid hybridization formats are known to those skilled in the art. For example, common formats include sandwich assays and competition or displacement assays. Hybridization techniques are generally described in Hames and Higgins Nucleic Acid Hybridization, A Practical Approach, IRL Press (1985); Gall and Pardue, Proc. Natl. Acad. Sci. U.S.A., 63:378-383 (1969); and John et al. Nature, 223:582-587 (1969).

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal. The binding of the signal generation complex is also readily amenable to accelerations by exposure to ultrasonic energy.

The label may also allow indirect detection of the hybridization complex. For example, where the label is a hapten or antigen, the sample can be detected by using antibodies. In these systems, a signal is generated by attaching fluorescent or enzyme molecules to the antibodies or in some cases, by attachment to a radioactive label (see, e.g., Tijssen, “Practice and Theory of Enzyme Immuoassays,” Laboratory Techniques in Biochemistry and Molecular Biology, Burdon and van Knippenberg Eds., Elsevier (1985), pp. 9-20).

The probes are typically labeled either directly, as with isotopes, chromophores, lumiphores, chromogens, or indirectly, such as with biotin, to which a streptavidin complex may later bind. Thus, the detectable labels used in the assays of the present invention can be primary labels (where the label comprises an element that is detected directly or that produces a directly detectable element) or secondary labels (where the detected label binds to a primary label, e.g., as is common in immunological labeling). Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labeled probes or the like.

Other labels include, e.g., ligands that bind to labeled antibodies, fluorophores, chemiluminescent agents, enzymes, and antibodies that can serve as specific binding pair members for a labeled ligand. An introduction to labels, labeling procedures and detection of labels is found in Polak and Van Noorden Introduction to Immunocytochemistry, 2nd ed., Springer Verlag, NY (1997); and in Haugland Handbook of Fluorescent Probes and Research Chemicals, a combined handbook and catalogue Published by Molecular Probes, Inc. (1996).

In general, a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill in the art. Commonly, an optical image of a substrate comprising bound labeling moieties is digitized for subsequent computer analysis.

The amount of, for example, an RNA is measured by quantitating the amount of label fixed to the solid support by binding of the detection reagent. Typically, the presence of a modulator during incubation will increase or decrease the amount of label fixed to the solid support relative to a control incubation that does not comprise the modulator, or as compared to a baseline established for a particular reaction type. Means of detecting and quantitating labels are well known to those of skill in the art.

In some embodiments, the target nucleic acid or the probe is immobilized on a solid support. Solid supports suitable for use in the assays of the invention are known to those of skill in the art. As used herein, a solid support is a matrix of material in a substantially fixed arrangement.

A variety of automated solid-phase assay techniques are also appropriate. For instance, very large scale immobilized polymer arrays (VLSIPS™), i.e. Gene Chips or microarrays, available from Affymetrix, Inc. in Santa Clara, Calif. can be used to detect changes in expression levels of a plurality of genes involved in the same regulatory pathways simultaneously. See, Tijssen, supra., Fodor et al. (1991) Science, 251: 767-777; Sheldon et al. (1993) Clinical Chemistry 39(4): 718-719, and Kozal et al. (1996) Nature Medicine 2(7): 753-759. Similarly, spotted cDNA arrays (arrays of cDNA sequences bound to nylon, glass or another solid support) can also be used to monitor expression of a plurality of genes.

Typically, the array elements are organized in an ordered fashion so that each element is present at a specified location on the substrate. Because the array elements are at specified locations on the substrate, the hybridization patterns and intensities (which together create a unique expression profile) can be interpreted in terms of expression levels of particular genes and can be correlated with a particular disease or condition or treatment. See, e.g., Schena et al., Science 270: 467-470 (1995)) and (Lockhart et al., Nature Biotech. 14: 1675-1680 (1996)).

Hybridization specificity can be evaluated by comparing the hybridization of specificity-control polynucleotide sequences to specificity-control polynucleotide probes that are added to a sample in a known amount. The specificity-control target polynucleotides may have one or more sequence mismatches compared with the corresponding polynucleotide sequences. In this manner, whether only complementary target polynucleotides are hybridizing to the polynucleotide sequences or whether mismatched hybrid duplexes are forming is determined.

Hybridization reactions can be performed in absolute or differential hybridization formats. In the absolute hybridization format, polynucleotide probes from one sample are hybridized to the sequences in a microarray format and signals detected after hybridization complex formation correlate to polynucleotide probe levels in a sample. In the differential hybridization format, the differential expression of a set of genes in two biological samples is analyzed. For differential hybridization, polynucleotide probes from both biological samples are prepared and labeled with different labeling moieties. A mixture of the two labeled polynucleotide probes is added to a microarray. The microarray is then examined under conditions in which the emissions from the two different labels are individually detectable. Sequences in the microarray that are hybridized to substantially equal number of polynucleotide probes derived from both biological samples give a distinct combined fluorescence (Shalon et al. PCT publication WO95/35505). In some embodiments, the labels are fluorescent labels with distinguishable emission spectra, such as Cy3 and Cy5 fluorophores.

After hybridization, the microarray is washed to remove nonhybridized nucleic acids and complex formation between the hybridizable array elements and the polynucleotide probes is detected. Methods for detecting complex formation are well known to those skilled in the art. In some embodiments, the polynucleotide probes are labeled with a fluorescent label and measurement of levels and patterns of fluorescence indicative of complex formation is accomplished by fluorescence microscopy, such as confocal fluorescence microscopy.

In a differential hybridization experiment, polynucleotide probes from two or more different biological samples are labeled with two or more different fluorescent labels with different emission wavelengths. Fluorescent signals are detected separately with different photomultipliers set to detect specific wavelengths. The relative abundances/expression levels of the polynucleotide probes in two or more samples are obtained.

Typically, microarray fluorescence intensities can be normalized to take into account variations in hybridization intensities when more than one microarray is used under similar test conditions. In some embodiments, individual polynucleotide probe/target complex hybridization intensities are normalized using the intensities derived from internal normalization controls contained on each microarray.

Detection of nucleic acids can also be accomplished, for example, by using a labeled detection moiety that binds specifically to duplex nucleic acids (e.g., an antibody that is specific for RNA-DNA duplexes). One example uses an antibody that recognizes DNA-RNA heteroduplexes in which the antibody is linked to an enzyme (typically by recombinant or covalent chemical bonding). The antibody is detected when the enzyme reacts with its substrate, producing a detectable product. Coutlee et al. (1989) Analytical Biochemistry 181:153-162; Bogulavski (1986) et al. J. Immunol. Methods 89:123-130; Prooijen-Knegt (1982) Exp. Cell Res. 141:397-407; Rudkin (1976) Nature 265:472-473, Stollar (1970) PNAS 65:993-1000; Ballard (1982) Mol. Immunol. 19:793-799; Pisetsky and Caster (1982) Mol. Immunol. 19:645-650; Viscidi et al. (1988) J. Clin. Microbial. 41:199-209; and Kiney et al. (1989) J. Clin. Microbiol. 27:6-12 describe antibodies to RNA duplexes, including homo and heteroduplexes. Kits comprising antibodies specific for DNA:RNA hybrids are available, e.g., from Digene Diagnostics, Inc. (Beltsville, Md.).

In addition to available antibodies, one of skill in the art can easily make antibodies specific for nucleic acid duplexes using existing techniques, or modify those antibodies that are commercially or publicly available. In addition to the art referenced above, general methods for producing polyclonal and monoclonal antibodies are known to those of skill in the art (see, e.g., Paul (ed) Fundamental Immunology, Third Edition Raven Press, Ltd., NY (1993); Coligan Current Protocols in Immunology Wiley/Greene, NY (1991); Harlow and Lane Antibodies: A Laboratory Manual Cold Spring Harbor Press, NY (1989); Stites et al. (eds.) Basic and Clinical Immunology (4th ed.) Lange Medical Publications, Los Altos, Calif., and references cited therein; Goding Monoclonal Antibodies: Principles and Practice (2d ed.) Academic Press, New York, N.Y., (1986); and Kohler and Milstein Nature 256: 495-497 (1975)). Other suitable techniques for antibody preparation include selection of libraries of recombinant antibodies in phage or similar vectors (see, Huse et al. Science 246:1275-1281 (1989); and Ward et al. Nature 341:544-546 (1989)). Specific monoclonal and polyclonal antibodies and antisera will usually bind with a K_{D} of at least about 0.1 μM, preferably at least about 0.01 μM or better, and most typically and preferably, 0.001 μM or better.

The nucleic acids used in this invention can be either positive or negative probes. Positive probes bind to their targets and the presence of duplex formation is evidence of the presence of the target. Negative probes fail to bind to the suspect target and the absence of duplex formation is evidence of the presence of the target. For example, the use of a wild type specific nucleic acid probe or PCR primers may serve as a negative probe in an assay sample where only the nucleotide sequence of interest is present.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBA, Cangene, Mississauga, Ontario) and Q Beta Replicase systems. These systems can be used to directly identify mutants where the PCR or LCR primers are designed to be extended or ligated only when a selected sequence is present. Alternatively, the selected sequences can be generally amplified using, for example, nonspecific PCR primers and the amplified target region later probed for a specific sequence indicative of a mutation. It is understood that various detection probes, including Taqman and molecular beacon probes can be used to monitor amplification reaction products, e.g., in real time.

An alternative means for determining the level of expression of the nucleic acids of the present invention is in situ hybridization. In situ hybridization assays are well known and are generally described in Angerer et al., Methods Enzymol. 152:649-660 (1987). In an in situ hybridization assay, cells, preferentially human cells from the cerebellum or the hippocampus, are fixed to a solid support, typically a glass slide. If DNA is to be probed, the cells are denatured with heat or alkali. The cells are then contacted with a hybridization solution at a moderate temperature to permit annealing of specific probes that are labeled. The probes are preferably labeled with radioisotopes or fluorescent reporters.

Single nucleotide polymorphism (SNP) analysis is also useful for detecting differences between alleles of CX3CR1 or fractalkine genes. CX3CR1 or fractalkine linked SNPs are useful, for instance, for diagnosis of CX3CR1 or fractalkine-linked diseases (e.g., diabetes) in a patient. For example, if an individual carries at least one allele of a CX3CR1 or fractalkine-linked SNP, the individual is likely predisposed for one or more of those diseases. If the individual is homozygous for a disease-linked CX3CR1 or fractalkine SNP, the individual is particularly predisposed for CX3CR1 or fractalkine-linked disease (e.g., diabetes). In some embodiments, the SNP associated with the CX3CR1 or fractalkine-linked disease is located within 300,000; 200,000; 100,000; 75,000; 50,000; or 10,000 base pairs of a polynucleotide encoding CX3CR1 or fractalkine.

Various real-time PCR methods including, e.g., Taqman or molecular beacon-based assays (e.g., U.S. Pat. Nos. 5,210,015; 5,487,972; Tyagi et al., Nature Biotechnology 14:303 (1996); and PCT WO 95/13399 are useful to monitor for the presence of absence of a SNP. Additional SNP detection methods include, e.g., DNA sequencing, sequencing by hybridization, dot blotting, oligonucleotide array (DNA Chip) hybridization analysis, or are described in, e.g., U.S. Pat. No. 6,177,249; Landegren et al., Genome Research, 8:769-776 (1998); Botstein et al., Am J Human Genetics 32:314-331 (1980); Meyers et al., Methods in Enzymology 155:501-527 (1987); Keen et al., Trends in Genetics 7:5 (1991); Myers et al., Science 230:1242-1246 (1985); and Kwok et al., Genomics 23:138-144 (1994)

V. Immunological Detection of CX3CR1 or Fractalkine

In addition to the detection of CX3CR1 or fractalkine genes and gene expression using nucleic acid hybridization technology, one can also use immunoassays to detect CX3CR1 or fractalkine polypeptides. Immunoassays can be used to qualitatively or quantitatvely analyze CX3CR1 or fractalkine. A general overview of the applicable technology can be found in Harlow & Lane, Antibodies: A Laboratory Manual (1988).

A. Antibodies to Target Proteins or Other Immunogens

Methods for producing polyclonal and monoclonal antibodies that react specifically with a protein of interest or other immunogen are known to those of skill in the art (see, e.g., Coligan, supra; and Harlow and Lane, supra; Stites et al., supra and references cited therein; Goding, supra; and Kohler and Milstein Nature, 256:495-497 (1975)). Such techniques include antibody preparation by selection of antibodies from libraries of recombinant antibodies in phage or similar vectors (see, Huse et al., supra; and Ward et al., supra). For example, in order to produce antisera for use in an immunoassay, the protein of interest or an antigenic fragment thereof, is isolated as described herein. For example, a recombinant protein is produced in a transformed cell line. An inbred strain of mice or rabbits is immunized with the protein using a standard adjuvant, such as Freund's adjuvant, and a standard immunization protocol. Alternatively, a synthetic peptide derived from the CX3CR1 or fractalkine sequences disclosed herein is conjugated to a carrier protein and used as an immunogen.

Polyclonal sera are collected and titered against the immunogen in an immunoassay, for example, a solid phase immunoassay with the immunogen immobilized on a solid support. Polyclonal antisera with a titer of 10⁴ or greater are selected and tested for their crossreactivity against non-CX3CR1 or fractalkine proteins or even other homologous proteins from other organisms, using a competitive binding immunoassay. Specific monoclonal and polyclonal antibodies and antisera will usually bind with a K_{D} of at least about 0.1 mM, more usually at least about 1 μM, preferably at least about 0.1 μM or better, and most preferably, 0.01 μM or better.

A number of proteins of the invention comprising immunogens may be used to produce antibodies specifically or selectively reactive with the proteins of interest. Recombinant protein is the preferred immunogen for the production of monoclonal or polyclonal antibodies. Naturally occurring protein may also be used either in pure or impure form. Synthetic peptides made using the protein sequences described herein may also be used as an immunogen for the production of antibodies to the protein. Recombinant protein can be expressed in eukaryotic or prokaryotic cells and purified as generally described supra. The product is then injected into an animal capable of producing antibodies. Either monoclonal or polyclonal antibodies may be generated for subsequent use in immunoassays to measure the protein.

Methods of production of polyclonal antibodies are known to those of skill in the art. In brief, an immunogen, preferably a purified protein, is mixed with an adjuvant and animals are immunized. The animal's immune response to the immunogen preparation is monitored by taking test bleeds and determining the titer of reactivity to CX3CR1 or fractalkine. When appropriately high titers of antibody to the immunogen are obtained, blood is collected from the animal and antisera are prepared. Further fractionation of the antisera to enrich for antibodies reactive to the protein can be done if desired (see, Harlow and Lane, supra).

Monoclonal antibodies may be obtained using various techniques familiar to those of skill in the art. Typically, spleen cells from an animal immunized with a desired antigen are immortalized, commonly by fusion with a myeloma cell (see, Kohler and Milstein, Eur. J. Immunol. 6:511-519 (1976)). Alternative methods of immortalization include, e.g., transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences that encode a monoclonal antibody or a binding fragment thereof by screening a DNA library from human B cells according to the general protocol outlined by Huse et al., supra.

Once target immunogen-specific antibodies are available, the immunogen can be measured by a variety of immunoassay methods with qualitative and quantitative results available to the clinician. For a review of immunological and immunoassay procedures in general see, Stites, supra. Moreover, the immunoassays of the present invention can be performed in any of several configurations, which are reviewed extensively in Maggio Enzyme Immunoassay, CRC Press, Boca Raton, Fla. (1980); Tijssen, supra; and Harlow and Lane, supra.

Immunoassays to measure target proteins in a human sample may use a polyclonal antiserum that was raised to the protein (e.g., CX3CR1 or fractalkine) or a fragment thereof. This antiserum is selected to have low cross-reactivity against non-CX3CR1 or fractalkine proteins and any such cross-reactivity is removed by immunoabsorption prior to use in the immunoassay.

B: Immunological Binding Assays

In some embodiments, a protein of interest is detected and/or quantified using any of a number of well-known immunological binding assays (see, e.g., U.S. Pat. Nos. 4,366,241; 4,376,110; 4,517,288; and 4,837,168). For a review of the general immunoassays, see also Asai Methods in Cell Biology Volume 37: Antibodies in Cell Biology, Academic Press, Inc. NY (1993); Stites, supra. Immunological binding assays (or immunoassays) typically utilize a “capture agent” to specifically bind to and often immobilize the analyte (in this case CX3CR1 or fractalkine of the present invention, or antigenic subsequences thereof). The capture agent is a moiety that specifically binds to the analyte. In a preferred embodiment, the capture agent is an antibody that specifically binds, for example, a CX3CR1 or fractalkine polypeptide of the invention. The antibody (e.g., anti-CX3CR1 or fractalkine antibody) may be produced by any of a number of means well known to those of skill in the art and as described above.

Immunoassays also often utilize a labeling agent to bind specifically to and label the binding complex formed by the capture agent and the analyte. The labeling agent may itself be one of the moieties comprising the antibody/analyte complex. Alternatively, the labeling agent may be a third moiety, such as another antibody, that specifically binds to the antibody/protein complex.

In a preferred embodiment, the labeling agent is a second antibody bearing a label. Alternatively, the second antibody may lack a label, but it may, in turn, be bound by a labeled third antibody specific to antibodies of the species from which the second antibody is derived. The second antibody can be modified with a detectable moiety, such as biotin, to which a third labeled molecule can specifically bind, such as enzyme-labeled streptavidin.

Other proteins capable of specifically binding immunoglobulin constant regions, such as protein A or protein G, can also be used as the label agents. These proteins are normal constituents of the cell walls of streptococcal bacteria. They exhibit a strong non-immunogenic reactivity with immunoglobulin constant regions from a variety of species (see, generally, Kronval, et al. J Immunol., 111: 1401-1406 (1973); and Akerstrom, et al. J Immunol., 135:2589-2542 (1985)).

Throughout the assays, incubation and/or washing steps may be required after each combination of reagents. Incubation steps can vary from about 5 seconds to several hours, preferably from about 5 minutes to about 24 hours. The incubation time will depend upon the assay format, analyte, volume of solution, concentrations, and the like. Usually, the assays will be carried out at ambient temperature, although they can be conducted over a range of temperatures, such as 10° C. to 40° C.

1. Non-Competitive Assay Formats

Immunoassays for detecting proteins or analytes of interest from tissue samples may be either competitive or noncompetitive. Noncompetitive immunoassays are assays in which the amount of captured protein or analyte is directly measured. In one preferred “sandwich” assay, for example, the capture agent (e.g. CX3CR1 or fractalkine antibodies) can be bound directly to a solid substrate where it is immobilized. These immobilized antibodies then capture the CX3CR1 or fractalkine present in the test sample. The CX3CR1 or fractalkine thus immobilized is then bound by a labeling agent, such as a second anti-CX3CR1 or fractalkine antibody bearing a label. Alternatively, the second antibody may lack a label, but it may, in turn, be bound by a labeled third antibody specific to antibodies of the species from which the second antibody is derived. The second can be modified with a detectable moiety, such as biotin, to which a third labeled molecule can specifically bind, such as enzyme-labeled streptavidin.

2. Competitive Assay Formats

In competitive assays, the amount of protein or analyte present in the sample is measured indirectly by measuring the amount of an added (exogenous) protein or analyte (e.g., the CX3CR1 or fractalkine of interest) displaced (or competed away) from a specific capture agent (e.g., antibodies raised to CX3CR1 or fractalkine) by the protein or analyte present in the sample. The amount of immunogen bound to the antibody is inversely proportional to the concentration of immunogen present in the sample. In a particularly preferred embodiment, the antibody is immobilized on a solid substrate. The amount of analyte may be detected by providing a labeled analyte molecule. It is understood that labels can include, e.g., radioactive labels as well as peptide or other tags that can be recognized by detection reagents such as antibodies.

Immunoassays in the competitive binding format can be used for cross-reactivity determinations. For example, the protein encoded by the sequences described herein can be immobilized on a solid support. Proteins are added to the assay and compete with the binding of the antisera to the immobilized antigen. The ability of the above proteins to compete with the binding of the antisera to the immobilized protein is compared to that of the protein encoded by any of the sequences described herein. The percent cross-reactivity for the above proteins is calculated, using standard calculations. Those antisera with less than 10% cross-reactivity with each of the proteins listed above are selected and pooled. The cross-reacting antibodies are optionally removed from the pooled antisera by immunoabsorption with the considered proteins, e.g., distantly related homologs.

The immunoabsorbed and pooled antisera are then used in a competitive binding immunoassay as described above to compare a second protein, thought to be perhaps a protein of the present invention, to the immunogen protein. In order to make this comparison, the two proteins are each assayed at a wide range of concentrations and the amount of each protein required to inhibit 50% of the binding of the antisera to the immobilized protein is determined. If the amount of the second protein required is less than 10 times the amount of the protein partially encoded by a sequence herein that is required, then the second protein is said to specifically bind to an antibody generated to an immunogen consisting of the target protein.

3. Other Assay Formats

In a particularly preferred embodiment, western blot (immunoblot) analysis is used to detect and quantify the presence of a CX3CR1 or fractalkine of the invention in the sample. The technique generally comprises separating sample proteins by gel electrophoresis on the basis of molecular weight, transferring the separated proteins to a suitable solid support (such as, e.g., a nitrocellulose filter, a nylon filter, or a derivatized nylon filter) and incubating the sample with the antibodies that specifically bind the protein of interest. For example, the anti-CX3CR1 or fractalkine antibodies specifically bind to the CX3CR1 or fractalkine on the solid support. These antibodies may be directly labeled or alternatively may be subsequently detected using labeled antibodies (e.g., labeled sheep anti-mouse antibodies) that specifically bind to the antibodies against the protein of interest.

Other assay formats include liposome immunoassays (LIA), which use liposomes designed to bind specific molecules (e.g., antibodies) and release encapsulated reagents or markers. The released chemicals are then detected according to standard techniques (see, Monroe et al. (1986) Amer. Clin. Prod. Rev. 5:3441).

4. Labels

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most labels useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g. polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, the ease of conjugation with the compound, stability requirements, available instrumentation, and disposal provisions.

Non-radioactive labels are often attached by indirect means. The molecules can also be conjugated directly to signal generating compounds, e.g., by conjugation with an enzyme or fluorescent compound. A variety of enzymes and fluorescent compounds can be used with the methods of the present invention and are well-known to those of skill in the art (for a review of various labeling or signal producing systems which may be used, see, e.g., U.S. Pat. No. 4,391,904).

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally simple colorimetric labels may be detected directly by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need to be labeled and the presence of the target antibody is detected by simple visual inspection.

VI. Identification of Modulators of CX3CR1 or Fractalkine

Modulators of CX3CR1 or fractalkine, i.e. agonists or antagonists of CX3CR1 or fractalkine activity, or CX3CR1 or fractalkine polypeptide or polynucleotide expression, are useful for treating a number of human diseases, including diabetes. For example, administration of CX3CR1 agonists can be used to treat diabetic patients or individuals with insulin resistance to prevent progression, and therefore symptoms, associated with diabetes.

A. Agents that Modulate CX3CR1 or Fractalkine

The agents tested as modulators of CX3CR1 or fractalkine can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds that can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). Modulators also include agents designed to reduce the level of CX3CR1 or fractalkine mRNA (e.g. antisense molecules, ribozymes, DNAzymes, small inhibitory RNAs and the like) or the level of translation from an mRNA (e.g., translation blockers such as an antisense molecules that are complementary to translation start or other sequences on an mRNA molecule). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential modulator compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g. Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

B. Methods of Screening for Modulators of CX3CR1 or Fractalkine

A number of different screening protocols can be utilized to identify agents that modulate the level of expression or activity of CX3CR1 or fractalkine in cells, particularly mammalian cells, and especially human cells. In general terms, the screening methods involve screening a plurality of agents to identify an agent that modulates the activity of CX3CR1 or fractalkine by, e.g., binding to a CX3CR1 or fractalkine polypeptide, preventing an inhibitor or activator from binding to CX3CR1 or fractalkine, increasing association of an inhibitor or activator with CX3CR1 or fractalkine, or activating or inhibiting expression of CX3CR1 or fractalkine. Wile screening methods for identifying modulators of CX3CR1 or fractalkine are discussed together, it is understood that the methods are typically employed to screen for either CX3CR1 or fractalkine. The two targets are discussed together for convenience.

In some embodiments, the modulators of the invention are specific for CX3CR1, fractalkine, or both. For example, in some embodiments, the modulators do not modulate the activity of other chemokines or chemokine receptors.

Any cell expressing CX3CR1 or a fragment thereof can be used to identify modulators. In some embodiments, the cells are eukaryotic cells lines (e.g., CHO or HEK293) transformed to express a heterologous CX3CR1 polypeptide. In some embodiments, a cell expressing an endogenous CX3CR1 is used in screens. In some embodiments, the screens are performed in the absence of insulin. In other embodiments, modulators are screened for their ability to effect insulin responses.

1. CX3CR1 or Fractalkine Binding Assays

Preliminary screens can be conducted by screening for agents capable of binding to CX3CR1 or fractalkine, as at least some of the agents so identified are likely CX3CR1 or fractalkine modulators. Binding assays are also useful, e.g., for identifying endogenous proteins that interact with CX3CR1 or fractalkine. For example, antibodies, receptors or other molecules that bind CX3CR1 or fractalkine can be identified in binding assays.

Binding assays usually involve contacting a CX3CR1 or fractalkine protein with one or more test agents and allowing sufficient time for the protein and test agents to form a binding complex. Any binding complexes formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to CX3CR1 or fractalkine or displacement of labeled substrates. The CX3CR1 or fractalkine proteins utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a host cell.

2. CX3CR1 Activity

CX3CR1 and its alleles and polymorphic variants are G-protein coupled receptors that participate in ligand-triggered signal transduction. The activity of CX3CR1 polypeptides can be assessed using a variety of in vitro and in vivo assays to determine functional, chemical, and physical effects, e.g., measuring ligand binding (e.g., radioactive or otherwise labeled ligand binding), second messengers (e.g., cAMP, cGMP, IP₃, DAG, or Ca²⁺), ion flux, phosphorylation levels, transcription levels, and the like. Furthermore, such assays can be used to test for inhibitors and activators of CX3CR1. Modulators can also be genetically altered versions of CX3CR1. Such modulators of transduction activity are useful for controlling cell signaling and treating diabetes (e.g., by stimulating CX3CR1 activity).

The CX3CR1 of the assay will be selected from a polypeptide having a sequence of SEQ ID NOS:8, 10 or 12 or conservatively modified variant thereof. Alternatively, the CX3CR1 of the assay will be derived from a eukaryote and include an amino acid subsequence having amino acid sequence identity to SEQ ID NOS:8, 10 or 12. Generally, the amino acid sequence identity will be at least 70%, optionally at least 85%, optionally at least 90-95%. Optionally, the polypeptide of the assays will comprise a fragment of CX3CR1 comprising one or more domains of CX3CR1, such as an extracellular domain, transmembrane domain, cytoplasmic domain, ligand binding domain, subunit association domain, active site, and the like. Either CX3CR1 or a domain thereof can be covalently linked to a heterologous protein to create a chimeric protein used in the assays described herein.

Modulators of CX3CR1 activity are tested using CX3CR1 polypeptides as described above, either recombinant or naturally occurring. The protein can be isolated, expressed in a cell, expressed in a membrane derived from a cell, expressed in tissue or in an animal, either recombinant or naturally occurring. For example, tissue slices, dissociated cells, e.g., from tissues expressing CX3CR1, transformed cells, or membranes can be used. Modulation is tested using one of the in vitro or in vivo assays described herein. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and or cytoplasmic domain of a receptor. Furthermore, ligand-binding domains of the protein of interest can be used in vitro in soluble or solid state reactions to assay for ligand binding.

Ligand binding to CX3CR1, a domain, or chimeric protein can be tested in solution, in a bilayer membrane, attached to a solid phase, in a lipid monolayer, or in vesicles. Binding of a modulator can be tested using, e.g., changes in spectroscopic characteristics (e.g., fluorescence, absorbance, refractive index), hydrodynamic (e.g., shape), chromatographic, or solubility properties.

Receptor-G-protein interactions can also be examined. Binding of the G-protein to the receptor or its release from the receptor can also be examined. For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998). In the absence of ligand or an agonist, there is a tight association between the heterotrimeric G protein and the GPCR. Stimulation by either the endogenous ligand (fractalkine) or an agonist of CX3CR1 will allow a conformational change that leads to the exchange of GDP to GTP on the α subunit of the trimeric G protein complex. This results in dissociation of the α subunit from the βγ subunits therby triggering multiple downstream signals.

These signal transduction events include activation of phospholipases, hydrolysis of phosphatidylinositol (4,5) bisphosphate, formation of diacylglycerol and inositol trisphosphate (IP3), intracellular calcium influx and activation of protein kinase C (PKC), AKT/PKB, and Mitogen-activated protein kinases (MAPK). Heterodimer βγG subunits have been reported to activate p110γ PI3-kinase dependent signalling. Gβγ-p110γ PI3-kinase dependent signals play a role in the process of activating AKT/PKB, p42/44 MAPK as well as the calcium influx.

There is considerable evidence that both the active GTP bound form of the α subunit as well as the βγ subunit of the G protein can activate these signals. The downstream signalling for CX3CR1 appears to be pertussis toxin sensitive, suggesting that the G protein belongs to a Gαi class. Activation of Gαi will result in inhibition of adenylate cyclase and therefore decreases intracellular concentrations of cyclic AMP. It has been clearly demonstrated that pretreatment of freshly isolated rat adipocytes and soleus muscle with pertussis toxin decreases insulin stimulated glucose uptake (Kanoh et al., Cell Signal: 12:223-232 (2000)). Studies with a transgenic mice overexpressing a constitutive active form of a Gαi subunit in skeletal muscle, adipose and liver tissues displayed increased glucose transport and translocation of GLUT4 to the plasma membrane in the absence of insulin in vivo (Song et al., J. Biol. Chem. 276:34651 (2001)). These systems can be adapted to identify CX3CR1 or fractalkine modulators whose downstream effect is to regulate glucose transport.

Compounds that modulate the duration of signal transduction are useful, for example, to treat diabetes or pre-diabetic individuals. Activated GPCR receptors become substrates for G-protein coupled receptor kinases (GRKs) that phosphorylate the C-terminal tail of the receptor (and possibly other sites as well). Thus, activators can promote the transfer of ³²P from gamma-labeled ATP to the receptor, which can be assayed with a scintillation counter. The phosphorylation of the C-terminal tail will promote the binding of arrestin-like proteins and will interfere with the binding of G-proteins. The GRK/β-arrestins pathway plays a role in the desensitization of many GPCR receptors, potentially regulating duration of signal transduction. In addition to their role as GPCR-specific endocytic adaptor proteins, β-arrestins also serve as molecular scaffolds that foster the formation of alternative, heterotrimeric G protein-independent signal transduction complexes. β-arrestin has been found to interact with several proteins including the nonreceptor tyrosine kinase c-src, ASK1 and JNK3 (see, e.g., Ferguson, Pharmacological Reviews 53:1-24 (2001) and Miller & Lefkowitz, Curr. Opin. Cell Biol., 13:139 (2001)).

In addition, a number of kinases that are phosphorylated and upregulated in response to CX3CR1 stimulation can be analyzed to indirectly measure fractalkine and/or CX3CR1 activity. Examples of such kinases include, e.g., AKT/PKB (see, e.g., Meucci et al., Proc. Natl. Acad. Sci. USA 97:8075 (2000)), MAPK (see, e.g., Cambien et al., Blood 97:2031-2037 (2001)), SAPK/JNK1 and p38/SAPK2 (see, e.g., Cambien et al., Blood 97:2031-2037 (2001)) and nonreceptor tyrosine kinases such as p60 src and p72 syk (see, e.g., Cambien et al., Blood 97:2031-2037 (2001)). The phosphorylation state of any of these proteins can be detected and quantified according to known methods, e.g., using phosph-specific antibodies in ELISA assays. Similarly, the kinase activity of these proteins can be detected using standard kinase assays, for example, using generic in vitro substrates such as myelin basic protein. In addition, reporter gene assays can be used to monitor activation of transcriptional complexes regulated by the above-described proteins (e.g., MAPK). Thus, modulation of the phosphorylation state or kinase activity of down stream proteins in response to small molecule treatment of a cell indicates that the small molecule modulates CX3CR1 activity.

One method for high throughput identification of molecules that modulate CX3CR1-dependent phosphorylation involves translational fusions of the DNA binding domain of a first transcription factor and the regulatory domain of a second transcription factor. See, e.g., Rees, et al, J. Biomolecular Screening 6(1):19-27 (2001). For example, cells expressing CX3CR1 are transfected with a fusion of the regulatory domain of a mammalian transcription factor (e.g., amino acids 83431 of SAP1) and the DNA binding and transcriptional activation domain of yeast GAL4. The fusion recognizes and activates a reporter gene fusion construct when the mammalian transcription factor domain is activated by MAPK as an indirect measurement of CX3CR1 activity.

Any suitable physiological change that affects GPCR activity can also be used to assess the influence of a test compound on the polypeptides of this invention. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺ (see, e.g., Kansra et al., J. Biol. Chem. 276:31831-31838 (2001)), IP3 (see, e.g., J. Biol. Chem. 274:10053-10058 (1999) and Berridge & Irvine, Nature 312:315-21 (1984)) or cAMP. Cells expressing G-protein coupled receptors such as CX3CR1 may exhibit increased cytoplasmic calcium levels as a result of activation of ion channels.

Preferred assays for G-protein coupled receptors include cells that are loaded with ion or voltage sensitive dyes to report receptor activity. Assays for determining activity of such receptors can also use known agonists and antagonists of CX3CR1 or other G-protein coupled receptors as negative or positive controls to assess activity of tested compounds. In assays for identifying modulatory compounds (e.g., agonists, antagonists), changes in the level of ions in the cytoplasm or membrane voltage will be monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wimlie et al., Proc. Nat'l Acad. Sci. USA 88:10049-10053-(1991)). Such promiscuous G-proteins allow coupling of a wide range of receptors.

Changes in ion flux may also be assessed by determining changes in polarization (i.e., electrical potential) of the cell or membrane expressing CX3CR1. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J Membrane Biology 137:59-70 (1994)).

Other assays can involve determining the activity of receptors which, when activated, result in a change in the level of intracellular cyclic nucleotides, e.g., cAMP or cGMP, by activating or inhibiting enzymes such as adenylate cyclase. There are cyclic nucleotide-gated ion channels, e.g., rod photoreceptor cell channels and olfactory neuron channels that are permeable to cations upon activation by binding of cAMP or cGMP (see, e.g., Altenhofen et al., Proc. Natl. Acad. Sci. U.S.A. 88:9868-9872 (1991) and Dhallan et al., Nature 347:184-187 (1990)). In cases where activation of the receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-gated ion channel, GPCR phosphalase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, the changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for 48 or more hrs. The labeled cells are treated with a test compound for one hour. The treated cells are lysed and extracted in chloroform-methanol-water after which the inositol phosphates were separated by ion exchange chromatography and quantified by scintillation counting. Fold stimulation is determined by calculating the ratio of cpm in the presence of agonist to cpm in the presence of buffer control. Likewise, fold inhibition is determined by calculating the ratio of cpm in the presence of antagonist to cpm in the presence of buffer control (which may or may not contain an agonist).

Other assays to determine CX3CR1 or fractalkine function include, e.g., chemotaxis assays or exocytosis assays to determine the effect of modulators on granulation and granule release of serine esterases. See, e.g., PCT/US00/23837.

Samples or assays that are treated with a potential CX3CR1 inhibitor or activator are compared to control samples without the test compound, to examine the extent of modulation. Control samples (untreated with activators or inhibitors) are assigned a relative CX3CR1 activity value of 100. Inhibition of CX3CR1 is achieved when the CX3CR1 activity value relative to the control is about 90%, optionally 50%, optionally 25-0%. Activation of CX3CR1 is achieved when the CX3CR1 activity value relative to the control is 110%, optionally 150%, 200%, 300%, 400%, 500%, or 1000-2000%.

3. Expression Assays

Screening for a compound that modulates the expression of CX3CR1 or fractalkine are also provided. Screening methods generally involve conducting cell-based assays in which test compounds are contacted with one or more cells expressing CX3CR1 or fractalkine, and then detecting an increase or decrease in CX3CR1 or fractalkine expression (either transcript or translation product). Assays can be performed with any cells that express CX3CR1 or fractalkine.

CX3CR1 or fractalkine expression can be detected in a number of different ways. As described infra, the expression level of CX3CR1 or fractalkine in a cell can be determined by probing the mRNA expressed in a cell with a probe that specifically hybridizes with a transcript (or complementary nucleic acid derived therefrom) of CX3CR1 or fractalkine. Probing can be conducted by lysing the cells and conducting Northern blots or without lysing the cells using in situ-hybridization techniques. Alternatively, CX3CR1 or fractalkine protein can be detected using immunological methods in which a cell lysate is probed with antibodies that specifically bind to CX3CR1 or fractalkine.

Other cell-based assays involve reporter assays conducted with cells using standard reporter gene assays. These assays can be performed in either cells that do, or do not, express CX3CR1 or fractalkine. Some of these assays are conducted with a heterologous nucleic acid construct that includes a CX3CR1 or fractalkine promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2: 101).

In these assays, cells harboring the reporter construct are contacted with a test compound. Modulated promoter expression is monitored by detecting the level of a detectable reporter. A number of different kinds of CX3CR1 or fractalkine modulators can be identified in this assay. For example, a test compound that inhibits the promoter by binding to it, inhibits the promoter by binding to transcription factors or other regulatory factors, binds to their promoter or triggers a cascade that produces a molecule that inhibits the promoter can be identified. Similarly a test compound that, e.g., activates the promoter by binding to it, activates the promoter by binding to transcription factors or other regulatory factors, binds to their promoter or triggers a cascade that produces a molecule that activates the promoter can also be identified.

The level of expression or activity can be compared to a baseline value. The baseline value can be a value for a control sample or a statistical value that is representative of CX3CR1 or fractalkine expression levels for a control population (e.g., lean individuals as described herein) or cells (e.g., tissue culture cells not exposed to an CX3CR1 or fractalkine modulator). Expression levels can also be determined for cells that do not express CX3CR1 or fractalkine as a negative control. Such cells generally are otherwise substantially genetically the same as the test cells.

A variety of different types of cells can be utilized in the reporter assays. Cells that express an endogenous CX3CR1 include, e.g., monocytes, neutrophils, leukocytes or brain, spleen cells, skeletal muscle or adipocytes. Cells that do not endogenously express CX3CR1 can be prokaryotic, but are preferably eukaryotic. The eukaryotic cells can be any of the cells typically utilized in generating cells that harbor recombinant nucleic acid constructs. Exemplary eukaryotic cells include, but are not limited to, yeast, and various higher eukaryotic cells such as the HEK293, HepG2, COS, CHO and HeLa cell lines.

Various controls can be conducted to ensure that an observed activity is authentic including running parallel reactions with cells that lack the reporter construct or by not contacting a cell harboring the reporter construct with test compound. Compounds can also be further validated as described below.

4. Validation

Agents that are initially identified by any of the foregoing screening methods can be further tested to validate the apparent activity. Modulators which are selected for further study can be tested on the “classic” insulin responsive cell line, mouse 3T3-L1 adipocytes. Adipocytes are pre-incubated with the modulators and tested for acute (up to 4 hours) and chronic (overnight) effects on basal and insulin-stimulated GLUT4 translocation and glucose uptake.

Following such studies, validity of the modulators is tested in suitable animal models. The basic format of such methods involves administering a lead compound identified during an initial screen to an animal that serves as a model for humans and then determining if CX3CR1 is in fact modulated.

The effect of the compound will be assessed in either diabetic animals or in diet induced insulin resistant animals. The blood glucose and insulin levels will be determined. The animal models utilized in validation studies generally are mammals of any kind. Specific examples of suitable animals include, but are not limited to, primates, mice and rats. For example, monogenic models of diabetes (e.g., ob/ob and db/db mice, Zucker rats and Zucker Diabetic Fatty rats etc) or polygenic models of diabetes (e.g., OLETF rats, GK rats, NSY mice, and KK mice) can be useful for validating CX3CR1 or fractalkine modulation in a diabetic or insulin resistant animals. In addition, transgenic animals expressing human CX3CR1 can be used to further validate drug candidates.

C. Solid Phase and Soluble High Throughput Assays

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100 to about 1500 different compounds. It is possible to assay several different plates per day; assay screens for up to about 6,000-20,000 or more different compounds are possible using the integrated systems of the invention. In addition, microfluidic approaches to reagent manipulation can be used.

The molecule of interest (e.g., CX3CR1) can be bound to the solid state component, directly or indirectly, via covalent or non-covalent linkage, e.g., via a tag. The tag can be any of a variety of components. In general, a molecule that binds the tag (a tag binder) is fixed to a solid support, and the tagged molecule of interest (e.g., CX3CR1) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, poly-His, etc.) Antibodies to molecules with natural binders such as biotin are also widely available and appropriate tag binders (see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs, such as agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I (1993)). Similarly, toxins and venoms, viral epitopes, hormones (e.g., opiates, steroids, etc.), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-Gly sequences of between about 5 and 200 amino acids (SEQ ID NO:15). Such flexible linkers are known to those of skill in the art. For example, poly(ethylene glycol) linkers are available from Shearwater Polymers, Inc., Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature (see, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank and Doring, Tetrahedron 44:60316040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

The invention provides in vitro assays for identifying, in a high throughput format, compounds that can modulate the expression or activity of CX3CR1. Control reactions that measure CX3CR1 activity of the cell in a reaction that does not include a potential modulator are optional, as the assays are highly uniform. Such optional control reactions are appropriate and increase the reliability of the assay. Accordingly, in a preferred embodiment, the methods of the invention include such a control reaction. For each of the assay formats described, “no modulator” control reactions that do not include a modulator provide a background level of binding activity.

In some assays it will be desirable to have positive controls. At least two types of positive controls are appropriate. First, a known activator of CX3CR1 of the invention can be incubated with one sample of the assay, and the resulting increase in signal resulting from an increased expression level or activity of CX3CR1 are determined according to the methods herein. Second, a known inhibitor of CX3CR1 can be added, and the resulting decrease in signal for the expression or activity of CX3 CR1 can be similarly detected. It will be appreciated that modulators can also be combined with activators or inhibitors to find modulators that inhibit the increase or decrease that is otherwise caused by the presence of the known modulator of CX3CR1.

VII. Compositions, Kits and Integrated Systems

The invention provides compositions, kits and integrated systems for practicing the assays described herein using nucleic acids-encoding the CX3CR1 or fractalkine polypeptides of the invention, or CX3CR1 or fractalkine proteins, anti-CX3CR1 or fractalkine antibodies, etc.

The invention provides assay compositions for use in solid phase assays; such compositions can include, for example, one or more nucleic acids encoding a CX3CR1 or fractalkine immobilized on a solid support, and a labeling reagent. In each case, the assay compositions can also include additional reagents that are desirable for hybridization. Modulators of expression or activity of a CX3CR1 or fractalkine of the invention can also be included in the assay compositions.

The invention also provides kits for carrying out the assays of the invention. The kits typically include a probe that comprises an antibody that specifically binds to CX3CR1 or fractalkine or a polynucleotide sequence encoding a CX3CR1 or fractalkine polypeptide, and a label for detecting the presence of the probe. The kits may include at least one polynucleotide sequence encoding a CX3CR1 or fractalkine polypeptides of the invention. Kits can include any of the compositions noted above, and optionally further include additional components such as instructions to practice a high-throughput method of assaying for an effect on expression of the genes encoding the CX3CR1 or fractalkine polypeptides of the invention, or on activity of the CX3CR1 polypeptides of the invention, one or more containers or compartments (e.g., to hold the probe, labels, or the like), a control modulator of the expression or activity of CX3CR1 or fractalkine polypeptides, a robotic armature for mixing kit components or the like.

The invention also provides integrated systems for high-throughput screening of potential modulators for an effect on the expression or activity of the CX3CR1 or fractalkine polypeptides of the invention. The systems can include a robotic armature which transfers fluid from a source to a destination, a controller which controls the robotic armature, a label detector, a data storage unit which records label detection, and an assay component such as a microtiter dish comprising a well having a reaction mixture or a substrate comprising a fixed nucleic acid or immobilization moiety.

A number of robotic fluid transfer systems are available, or can easily be made from existing components. For example, a Zymate XP (Zymark Corporation; Hopkinton, Mass.) automated robot using a Microlab 2200 (Hamilton; Reno, Nev.) pipetting station can be used to transfer parallel samples to 96 well microtiter plates to set up several parallel simultaneous binding assays.

Optical images viewed (and, optionally, recorded) by a camera or other recording device (e.g., a photodiode and data storage device) are optionally further processed in any of the embodiments herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS®, OS2® WINDOWS®, WINDOWS NT®, WINDOWS95®, WINDOWS98®, or WINDOWS2000′ based computers), MACINTOSH®, or UNIX® based (e.g., SUN® work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

VIII. Administration and Pharmaceutical Compositions

Modulators of CX3CR1 or fractalkine (e.g., antagonists or agonists) can be administered directly to the mammalian subject for modulation of CX3CR1 or fractalkine activity in vivo. Similarly, recombinant fractalkine or active fragments thereof (e.g., an active chemokine domain) can be administered. Administration is by any of the routes normally used for introducing a modulator compound into ultimate contact with the tissue to be treated and is well known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington's Pharmaceutical Sciences, 17^{th} ed. 1985)).

The modulators (e.g., agonists or antagonists) of the expression or activity of the CX3CR1 or fractalkine, alone or in combination with other suitable components, can be prepared for injection or for use in a pump device. Pump devices (also known as “insulin pumps”) are commonly used to administer insulin to patients and therefore can be easily adapted to include compositions of the present invention. Manufacturers of insulin pumps include Animas, Disetronic and MiniMed.

The modulators (e.g., agonists or antagonists) of the expression or activity of the CX3CR1 or fractalkine, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. In the practice of this invention, compositions can be administered, for example, orally, nasally, topically, intravenously, intraperitoneally, or intrathecally. The formulations of compounds can be presented in unit-dose or multi-dose sealed containers, such as ampoules and vials. Solutions and suspensions can be prepared from sterile powders, granules, and tablets of the kind previously described. The modulators can also be administered as part of a prepared food or drug.

The dose administered to a patient, in the context of the present invention should be sufficient to induce a beneficial response in the subject over time. The optimal dose level for any patient will depend on a variety of factors including the efficacy of the specific modulator employed, the age, body weight, physical activity, and diet of the patient, on a possible combination with other drugs, and on the severity of the case of diabetes. It is recommended that the daily dosage of the modulator be determined for each individual patient by those skilled in the art in a similar way as for known insulin compositions. The size of the dose also will be determined by the existence, nature, and extent of any adverse side-effects that accompany the administration of a particular compound or vector in a particular subject.

In determining the effective amount of the modulator to be administered a physician may evaluate circulating plasma levels of the modulator, modulator toxicity, and the production of anti-modulator antibodies. In general, the dose equivalent of a modulator is from about 1 ng/kg to 10 mg/kg for a typical subject.

For administration, CX3CR1 or fractalkine modulators of the present invention can be administered at a rate determined by the LD-50 of the modulator, and the side-effects of the modulator at various concentrations, as applied to the mass and overall health of the subject. Administration can be accomplished via single or divided doses.

The compounds of the present invention can also be used effectively in combination with one or more additional active agents depending on the desired target therapy (ee, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4). A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W., (ed.), Current Therapy In Endocrinology And Metabolism, 6th Edition (Mosby—Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al., Diabet. Med. (1996) 13 365-370; Kwiterovich, P. Am. J. Cardiol (1998) 82(12A): 3U-17U). These studies indicate that modulation of diabetes, among other diseases, can be further improved by the addition of a second agent to the therapeutic regimen. Combination therapy includes administration of a single pharmaceutical dosage formulation that contains a CX3CR1 or fractalkine modulator of the invention and one or more additional active agents, as well as administration of a CX3CR1 or fractalkine modulator and each active agent in its own separate pharmaceutical dosage formulation. For example, a CX3CR1 or fractalkine modulator and a thiazolidinedione can be administered to the human subject together in a single oral dosage composition, such as a tablet or capsule, or each agent can be administered in separate oral dosage formulations. Where separate dosage formulations are used, an CX3CR1 or fractalkine modulator and one or more additional active agents can be administered at essentially the same time (i.e., concurrently), or at separately staggered times (i.e., sequentially). Combination therapy is understood to include all these regimens.

One example of combination therapy can be seen in treating pre-diabetic individuals (e.g., to prevent progression into type 2 diabetes) or diabetic individuals (or treating diabetes and its related symptoms, complications, and disorders), wherein the CX3CR1 or fractalkine modulators can be effectively used in combination with, for example, sulfonylureas (such as chlorpropamide, tolbutamide, acetolhexamide, tolazamide, glyburide, gliclazide, glynase, glimepiride, and glipizide); biguanides (such as metformin); a PPAR beta delta agonist; a ligand or agonist of PPAR gamma such as thiazolidinediones (such as ciglitazone, pioglitazone (see, e.g., U.S. Pat. No. 6,218,409), troglitazone, and rosiglitazone (see, e.g. U.S. Pat. No. 5,859,037)); PPAR alpha agonists such as clofibrate, gemfibrozil, fenofibrate, ciprofibrate, and bezafibrate; dehydroepiandrosterone (also referred to as DHEA or its conjugated sulphate ester, DHEA-SO4); antiglucocorticoids; TNFα inhibitors; α-glucosidase inhibitors (such as acarbose, miglitol, and voglibose); amylin and amylin derivatives (such as pramlintide, (see, also, U.S. Pat. Nos. 5,902,726; 5,124,314; 5,175,145 and 6,143,718.)); insulin secretogogues (such as repaglinide, gliquidone, and nateglinide (see, also, U.S. Pat. Nos. 6,251,856; 6,251,865; 6,221,633; 6,174,856)), and insulin.

IX. Gene Therapy

Conventional viral and non-viral based gene transfer methods can be used to introduce nucleic acids encoding engineered polypeptides of the invention in mammalian cells or target tissues. Such methods can be used to administer nucleic acids encoding polypeptides of the invention (e.g., fractalkine or CX3CR1, including variants thereof) to cells in vitro. In some embodiments, the nucleic acids encoding polypeptides of the invention are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11: 162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids encoding engineered polypeptides of the invention take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SW), human immuno deficiency virus (HIV), and combinations thereof (see, e.g. Buchscher et al., J. Virol. 66,2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications where transient expression of the polypeptides of the invention is preferred, adenoviral based systems are typically used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994)). Construction of recombinant AAV vectors are described in a number of publications, including U.S. Pat. No. 5,173,414; Tratschin et al., Mol. Cell. Biol. 5:3251-3260 (1985); Tratschin, et al.; Mol. Cell. Biol. 4:2072-2081 (1984); Hermonat & Muzyczka, PNAS 81:6466-6470 (1984); and Samulski et al., J. Virol. 63:03822-3828 (1989).

pLASN and MFG-S are examples are retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)).

Replication-deficient recombinant adenoviral vectors (Ad) can be engineered such that a transgene replaces the Ad E1a, E1b, and E3 genes; subsequently the replication defector vector is propagated in human 293 cells that supply deleted gene function in trans. Ad vectors can transduce multiply types of tissues in vivo, including nondividing, differentiated cells such as those found in the liver, kidney and muscle system tissues. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:15-10 (1996); Sterman et al., Hum. Gene Ther. 9:7 1083-1089 (1998); Welsh et al., Hum. Gene Then. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and 42 cells or PA317 cells, which package retrovirus. Viral vectors used in gene therapy are usually generated by producer cell line that packages a nucleic acid vector into a viral particle. The vectors typically contain the minimal viral sequences required for packaging and subsequent integration into a host, other viral sequences being replaced by an expression cassette for the protein to be expressed. The missing viral functions are supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. A viral vector is typically modified to have specificity for a given cell type by expressing a ligand as a fusion protein with a viral coat protein on the viruses outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor., Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid (gene or cDNA) encoding a polypeptides of the invention, and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

Vectors (e.g., retroviruses, adenoviruses, liposomes, etc.) containing therapeutic nucleic acids can be also administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington's Pharmaceutical Sciences, 17th ed., 1989).

X. Diagnosis of Diabetes

The present invention also provides methods of diagnosing diabetes or a predisposition of at least some of the pathologies of diabetes. Diagnosis can involve determination of a genotype of an individual (e.g., with SNPs) and comparison of the genotype with alleles known to have an association with the occurrence of diabetes or other CX3CR1 or fractalkine-related disease. Alternatively, diagnosis also involves determining the level of CX3CR1 and/or fractalkine (protein or transcript) in a patient and then comparing the level to a baseline or range. Typically, the baseline value is representative of CX3CR1 or fractalkine in a healthy (e.g., lean) person.

As discussed above, variation of levels (e.g., high levels) of CX3CR1 or fractalkine from the baseline range indicates that the patient is either diabetic or at risk of developing at least some of the pathologies of diabetes (e.g., pre-diabetic). The level of a polypeptide in a lean individual can be a reading from a single individual, but is typically a statistically relevant average from a group of lean individuals. The level of a polypeptide in a lean individual can be represented by a value, for example in a computer program.

In some embodiments, the level of CX3CR1 or fractalkine are measured by taking a blood, urine or tissue sample from a patient and measuring the amount of CX3CR1 or fractalkine in the sample using any number of detection methods, such as those discussed herein. For instance, fasting and fed blood or urine levels can be tested.

In some embodiments, the baseline level and the level in a lean sample from an individual, or at least two samples from the same individual differ by at least about 5%, 10%, 20%, 50%, 75%, 100%, 150%, 200%, 300%, 400%, 500%, 1000% or more. In some embodiments, the sample from the individual is greater by at least one of the above-listed percentages relative to the baseline level. In some embodiments, the sample from the individual is lower by at least one of the above-listed percentages relative to the baseline level.

In some embodiments, the level of CX3CR1 or fractalkine is used to monitor the effectiveness of antidiabetic therapies such as thiazolidinediones, metformin, sulfonylureas and other standard therapies. In some embodiments the activity or expression of CX3CR1 or fractalkine will be measured prior to and after treatment of diabetic or pre-diabetic patients with antidiabetic therapies as a surrogate marker of clinical effectiveness. For example, the greater the reduction in CX3CR1 or fractalkine expression or activity indicates greater effectiveness.

Glucose/insulin tolerance tests can also be used to detect the effect of glucose levels on CX3CR1 or fractalkine levels. In glucose tolerance tests, the patient's ability to tolerate a standard oral glucose load is evaluated by assessing serum and urine specimens for glucose levels. Blood samples are taken before the glucose is ingested, glucose is given by mouth, and blood or urine glucose levels are tested at set intervals after glucose ingestion. Similarly, meal tolerance tests can also be used to detect the effect of insulin or food, respectively, on CX3CR1 or fractalkine levels.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

### EXAMPLES

The following examples are offered to illustrate, but not to limit the claimed invention.

This disclosure describes for the first time the involvement of CX3CR1 and its chemokine, fractalkine, in signaling and increasing insulin sensitivity in adipocytes and skeletal muscle.

Upregulation of CX3CR1 in Human Diabetic Skeletal Muscle as Determined by Affymetrix Genechips™

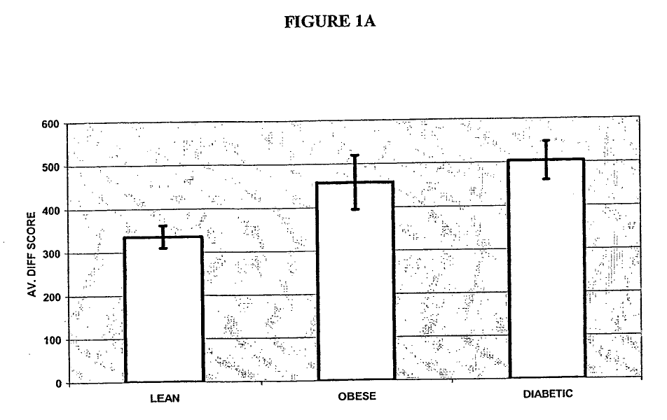

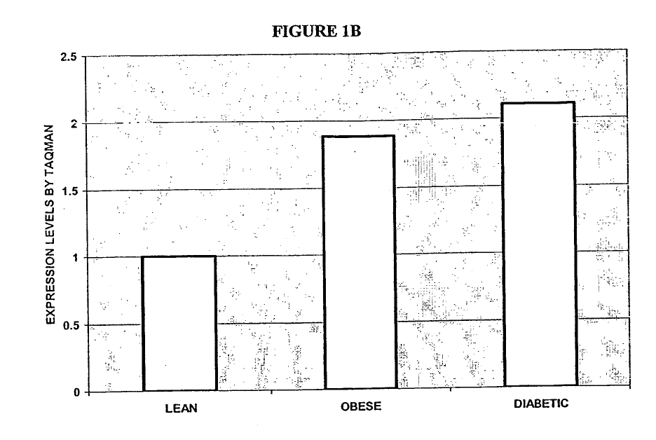

CX3CR1 was found to be upregulated 1.5 fold in diabetic subjects (DIABETIC) with statistical significance (students t-test p-value of <0.003) when compared to the lean non-diabetic values (LEAN). See, FIG. 1A. The mean average difference scores±standard error are 336±26 in the lean (n=17) and 503±44 in the diabetic (n=19) skeletal muscle samples after an overnight fast. An increase (a 1.36-fold upregulation) in CX3CR1 in the obese, non-diabetic insulin resistant subjects (OBESE, n=15) was also observed. The mean average difference score±standard error is 457±62 but lacked significance (students t-test with a p-value of <0.078).

Reconfirmation of Expression Levels of CX3CR1 by Quantitative PCR of Diabetic Skeletal Muscle

PCR primers and Taqman Probes were designed using Perkin Elmer's Primer Express software (Version 1.5). Briefly, primers were chosen to produce an amplicon of 80-120 nucleotides in length

| | |
|---|---|
| CX3CR1 6-fam-probe: | |
| AATGCCTGGCTGTCCTGTGTGGG; | (SEQ ID NO: 16) |
| CX3CR1 forward PCR primer: | |
| CAGAAGATACCTTTACCACCTGTATGG; | (SEQ ID NO: 17) |
| CX3CR1 reverse PCR primer: | |
| GAGGAGAAATCAACGTGGACTGA. | (SEQ ID NO: 18) |

Specificity was obtained by using primers and probes that hybridize only to human CX3CR1. CX3CR1 levels were 1.89- and 2.11-fold upregulated in obese and diabetic skeletal muscle, respectively, when compared to lean. See, FIG. 1B. The relative expression level as determined by quantitative PCR was 1.86, 3.51 and 3.92 in lean, obese and diabetic skeletal muscle samples, respectively.
Upregulation of CX3CR1 in Human Obese, Insulin Resistant Non-Diabetic Skeletal Muscle as Determined by Affymetrix Genechips™

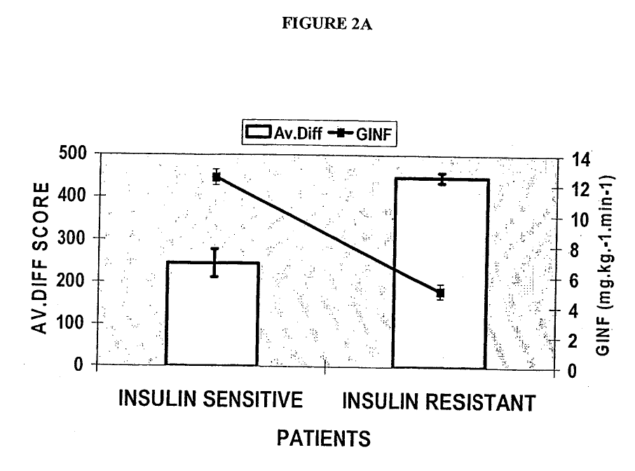

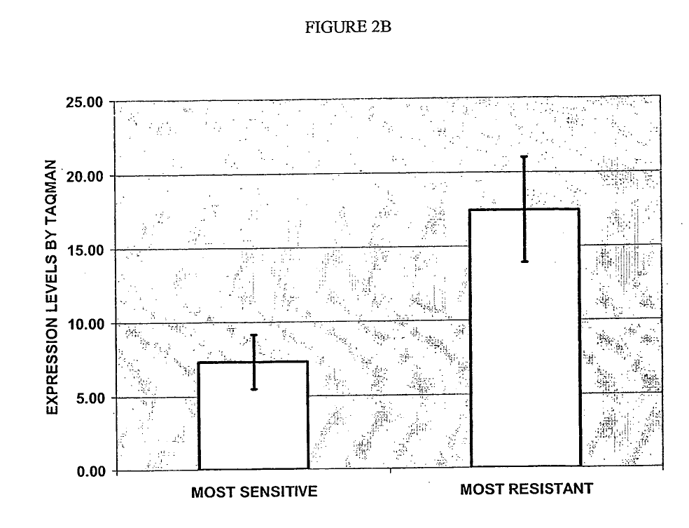

Comparison of the expression profiles between the 7 most lean/normal insulin sensitive individuals with the 7 most insulin resistant obese, non-diabetic individuals was studied. See FIG. 2A. This gene was upregulated 1.83 fold in the insulin resistant subjects with statistical significance (students test p-value<0.002). The mean average difference score (±standard error) for MBXHUMMUS03963 was 245±12 and 447±33 for insulin sensitive and insulin resistant samples, respectively. The expression levels of CX3CR1 negatively correlated to the insulin sensitivity of the subjects as measured by the glucose infusion rate into the skeletal muscle (GINF) (Ludvik et al., J. Clin. Invest. 100:2354 (1997); Frias et al., Diabetes Care 23:64, (2000)). The lean normal insulin sensitive population the GINF is 12.5±0.53 mg·kg-1·min-1 (n=7) whereas the obese non-diabetic subjects were insulin-resistant compared with lean normal subjects, with a GINF of 5.07±0.49 mg·kg-1·min-1 (n=7).

This data is consistent with the finding that CX3CR1 mRNA levels appear to have an inverse correlation to the degree of insulin resistance.

Reconfirmation of Uprepulated CX3CR1 Expression Levels by Quantitative PCR in Insulin Resistant Human Skeletal Muscle Tissue

CX3CR1 was 2.38-fold upregulated in insulin resistant skeletal muscle when compared to lean (p-value of <0.033). The mean relative expression levels±standard error as determined by quantitative PCR were 7.32±1.83 in lean normal insulin sensitive and 17.45±3.55 in insulin resistant obese non-diabetic skeletal samples. See FIG. 2B

Reconfirmation of Expression Levels of Fractalkine by Quantitative PCR of Diabetic Skeletal Muscle.

PCR primers and Taqman Probes were designed using Perkin Elmer's Primer Express software (Version 1.5). Briefly, primers were chosen to produce an amplicon of 80-120 nucleotides in length.

| | |
|---|---|
| Fractalkine 6-fam-probe: | |
| CAAACGCGCAATCATCTTGGAGACG; | (SEQ ID NO: 19) |
| Fractalkine forward PCR primer: | |
| TCAACAGAACCAGGCATCATG; | (SEQ ID NO: 20) |
| Fractalkine reverse PCR primer: | |
| CGGGTCGGCACAGAACAG. | (SEQ ID NO: 21) |

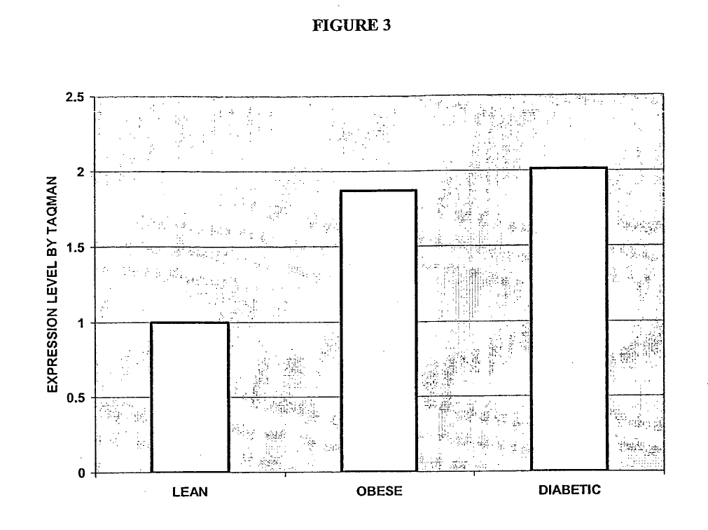

Specificity was obtained by using primers and probes that hybridize only to human fractalkine. Fractalkine levels were 1.47- and 1.84-fold upregulated in obese and diabetic skeletal muscle, respectively, when compared to lean. See, FIG. 3. The relative expression level as determined by quantitative PCR was 1.23, 1.81 and 2.26 in lean, obese and diabetic skeletal muscle samples, respectively.
Upregulation of CX3CR1 in a Mouse Model of Diet Induced Obesity and Insulin Resistance.

PCR primers were designed using Perkin Elmer's Primer Express software (Version 1.5). Briefly, primers were chosen to produce an amplicon of 80-120 nucleotides in length

| | |
|---|---|
| CX3CR1 forward PCR primer: | |
| AGAAGGGATACCTAAGATGCTGTTG; | (SEQ ID NO: 22) |
| CX3CR1 reverse PCR primer: | |
| CCACCCCCCAGCTTCTG. | (SEQ ID NO: 23) |

Specificity was obtained by using primers that hybridized only to mouse CX3CR1.

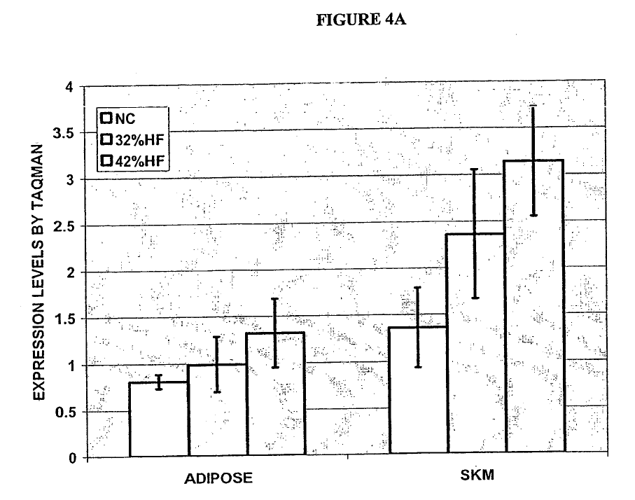

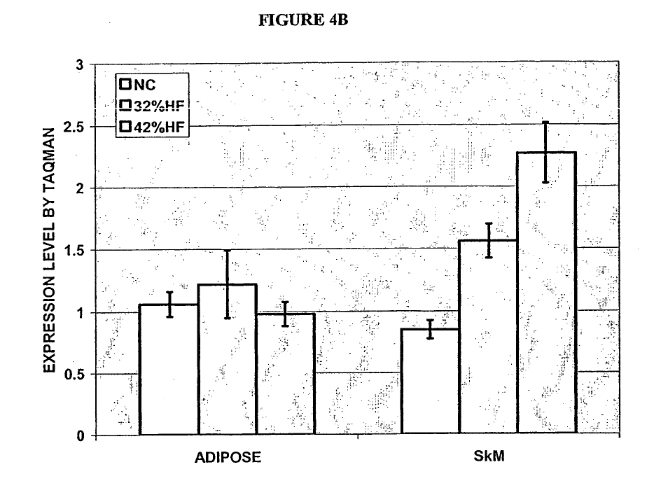

DB/JA mice were fed a normal chow (NC) or a chow with increasing fat content (32% and 42%) for 28 weeks. This mouse model of dietary induced obesity and insulin resistance is one of the best rodent models for mimicking human obese non-diabetic insulin resistance. We found the CX3CR1 to be 2.31-fold upregulated in skeletal muscle isolated from mice fed a 42% fat diet (HFD) when compared to normal chow (p-value of <0.04). See FIG. 4A. The mean relative expression levels±standard error (n=5) as determined by quantitative PCR were 0.81±0.075 in normal chow, 0.99±0.295 in 32% HFD and 1.32±0.367 in 42% HFD from epididymal adipose tissue, and, 1.36±0.426 in normal chow, 2.36±0.693 in 32% HFD and 3.14±0.586 in 42% HFD from skeletal muscle tissue.

Upregulation of Neurotactin in a Mouse Model of Diet Induced Obesity and Insulin Resistance

PCR primers were designed using Perkin Elmer's Primer Express software (Version 1.5). Briefly, primers were chosen to produce an amplicon of 80-120 nucleotides in length:

| | |
|---|---|
| Neurotactin forward PCR primer: | |
| CCAACTCCAGTGAACAATTATTTATTG; | (SEQ ID NO: 24) |
| Neuortactin reverse PCR primer: | |
| GCGCGCGGGAAACAG. | (SEQ ID NO: 25) |

Specificity was obtained by using primers that hybridize only to mouse neurotactin (mouse fractalkine).

DB/JA mice were fed a normal chow (NC) or a chow with increasing fat content (32% and 42%) for 28 weeks. The fractalkine levels were 1.85-fold (p-value<0.003) and 2.67-fold (P value<0.003) upregulated in skeletal muscle isolated from mice fed either a 32% or 42% fat diet (HFD) when compared to normal chow. See FIG. 4B. The mean relative expression levels±standard error (n=5) as determined by quantitative PCR were 1.06±0.101 in normal chow, 1.22±0.271 in 32% HFD and 0.98±0.097 in 42% HFD from epididymal adipose tissue, and, 0.85±0.074 in normal chow, 1.56±0.136 in 32% BFD and 2.27±0.243 in 42% HFD from skeletal muscle tissue. No effect by the diet on fractalkine expression levels was observed in samples prepared from the heart.

Fractalkine Induces Phosphorylation of AKT/PKB in the Absence of Insulin in 3T3-L1 Adipocytes

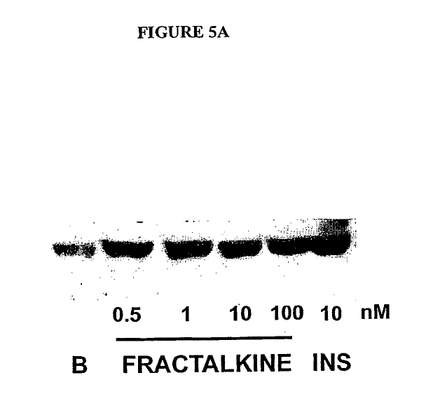

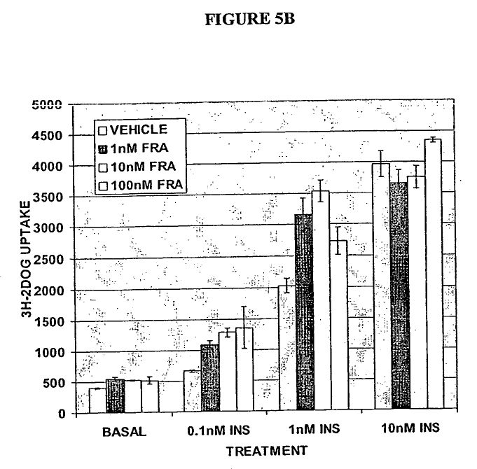

3T3-L1 adipocytes were pretreated with either vehicle alone (B), increasing concentrations of fractalkine (0.5, 1, 10 or 100 nM) for 30 minutes or with 10 nM insulin for 5 minutes at 37° C. Cells were washed once with cold PBS solution and then solubilized in lysis buffer. Equivalent amounts of total protein were separated on a 8% SDS-PAGE, transferred to PVDF membrane and immunoblotted with anti-phosphoserine 473-AKT/PKB rabbit polyclonal antibodies. Treatment of adipocytes with fractalkine increased the basal levels of AKT/PKB activity in the absence of insulin. See FIG. 5A.

Pretreatment of 3T3-L1 Adipocytes with Fractalkine Increases Insulin-Stimulated Glucose Uptake

3T3-L1 adipocytes were pretreated with either vehicle alone or recombinant soluble fractalkine (FRA) at the concentrations of 1, 10 or 100 nM for 4 hours at 37° C. The cells were then stimulated with insulin (INS) at basal, 0.1, 1, or 10 nM for 30 minutes. The adipocytes were assayed acutely for glucose transport activity by the measurement of the glucose transport using [3H]2-deoxy-glucose (2-DOG) uptake. Each assay was performed in triplicate. See FIG. 5B and Table 1.

FIG. 5B provides evidence that fractalkine through the activation of CX3CR1 can act as an insulin sensitizer. Fractalkine treatment alone had small but significant increases in basal glucose uptake levels, additive effects on sub-maximal insulin stimulated glucose uptake with no effect on maximal insulin stimulated glucose uptake into 3T3-L1 adipocytes. Thus, a conclusion from this data is that overexpression of CX3CR1 in insulin resistant or diabetic skeletal muscle represents one of the early compensatory events that occurs in an attempt to overcome the developing insulin resistant phenotype.

The data presented are mean 2-DOG uptake values±standard errors (n=3). The actual values are as follows:

Vehicle Alone+Basal 399±14, Vehicle Alone+0.1 nM INS 662±20,

Vehicle Alone+1 nM INS 2029±116, Vehicle Alone+10 nM INS 3972±212;

1 nM FRA+Basal 544±31, 1 nM FRA+0.1 nM INS 1091±62,

1 nM FRA+nM INS 3161±270, 1 nM FRA+10 nM INS 3659±206;

10 nM FRA+Basal 521±10, 10 nM FRA+0.1 nM INS 1282±76,

10 nM FRA+1 nM INS 3536±173, 10 nM FRA+10 nM INS 3753±180;

100 nM FRA+Basal 515±56, 100 nM FRA+0.1 nM INS 1354±339,

100 nM FRA+1 nM INS 2726±225; 100 nM FRA+10 nM INS 4352±43.

TABLE 1
Fold changes for the fractalkine induced increases in glucose uptake over vehicle pretreatment
alone determined from glucose uptake as shown in FIG. 5B. The Students t-test was used
to compare groups. p- values of ≦0.05 were considered significant.

| | | | +0.1 | | +1 nM | | +10 nM | |
|---|---|---|---|---|---|---|---|---|
| | BASAL | | nM INS | | INS | | INS | |
| | FC | pvalue | FC | pvalue | FC | pvalue | FC | pvalue |
| +1 nM FRA | 1.4 | 0.028 | 1.6 | 0.013 | 1.6 | 0.036 | 0.9 | 0.348 |
| +10 nM FRA | 1.3 | 0.003 | 1.9 | 0.01 | 1.8 | 0.003 | 0.9 | 0.475 |
| +100 nM FRA | 1.3 | 0.169 | 2 | 0.177 | 1.3 | 0.069 | 1.1 | 0.211 |

This study has defined the expression of both fractalkine and CX3CR1 in rodent and human samples of insulin resistance and/or diabetes. In obese, insulin resistant non-diabetic subjects, CX3CR1 gene levels were significantly increased over lean subjects. Furthermore this elevated gene expression level was observed in the diabetic population. The increased expression levels in insulin resistance were mimicked by treating mice with a high fat diet over 28 weeks. Without intending to limit the invention to a particular mechanism, it is believed that fractalkine acts as an insulin sensitizer indicating that overexpression of CX3CR1 is a compensatory mechanism and acts as an alternative means for the peripheral tissue to dispose of glucose.

CX3CR1 Gene Silencing in 3T3-L1 Adipocytes by Small Interfering RNAs Decreases Glucose Transport

Small interfering RNAs (siRNAs) (as described in, e.g., Elbashir, S. M. et al. Duplexes of 21-nucleotide RNAs mediate RNA interference in cultured mammalian cells. Nature 411:494-498 (2001)) were transfected into cultured adipocytes to induce specific degradation of the CX3CR1 messenger RNA in these cells. Insulin-stimulated 2-deoxyglocose uptake in adipocytes was assessed twenty four hours post transfection. Basal and insulin-stimulated 2-deoxyglucose uptake in cells transfected with siRNA 4 was significantly inhibited as compared with cells transfected with a scrambled siRNA at concentrations of 10 nM insulin (Table 2). CX3CR1 protein levels were evaluated by western blot and were found to be significantly decreased in cells transfected with siRNA4 (Table 3). A conclusion from these data is that siRNA-mediated attenuation of CX3CR1 expression causes a corresponding decrease in GLUT4 responsiveness to insulin. Thus, without intending to limit the invention to a particular mechanism of action, it can be inferred from these data that over-expression of CX3CR1 in insulin resistant or diabetic skeletal muscle represents one of the early compensatory events that occurs in an attempt to overcome the developing insulin resistant phenotype.

TABLE 2
Insulin-stimulated [³H]2-deoxyglucose (2DOG) uptake in differentiated
3T3-L1 adipocytes transfected with either a scrambled siRNA or siRNA
4. Data represent the average of three separate experiments and
are expressed as percents ± SD with the cpm values generated
for the scrambled siRNA Basal taken as 100%. The Students t-test
was used to compare groups (Scramble siRNA versus siRNA4 Basal;
Scramble siRNA versus siRNA4 Stimulated).

| | Basal (0 nM Insulin) | Stimulated (10 nM Insulin) |
|---|---|---|
| Scrambled siRNA | 100 ± 12 | 733.2 + 117 |
| siRNA 4 | 50.74** + 17 | 306.4** + 149 |

**P values of ≦0.05 were considered significant.

TABLE 3
CX3CR1 protein levels were evaluated by western blot using
a rabbit polyclonal antibody to rat CX3CR1 antibody (Abcam
ab7200). Quantification of the CX3CR1 protein was performed
using a scanning densitometer. Data are representative of
three separate experiments and are expressed as percents with
the arbitrary units values generated for the Scramble siRNA
taken as 100% for each individual experiments. The Students
t-test was used to compare groups. P values of ≦0.05 were
considered significant. N/A not applicable

| | % ± SD | p value |
|---|---|---|
| Scrambled siRNA | 100 ± 0 | |
| siRNA 4 | 46 ± 12 | 0.001 |

What is claimed is:

1. A method of identifying an agent for treating a diabetic or prediabetic patient, the method comprising the steps of:
(i) contacting a solution comprising an CX3CR1 polypeptide or ligand-binding fragment thereof with the agent, wherein the CX3CR1 polypeptide or ligand-binding fragment thereof is encoded by a nucleic acid that hybridizes under stringent conditions to a nucleic acid encoding a polypeptide having an amino acid sequence selected from the group consisting of SEQ ID NO:8, SEQ ID NO:10 and SEQ ID NO:12; and
(ii) selecting an agent that increases the expression or activity of the CX3CR1 polypeptide or ligand-binding fragment thereof, thereby identifying an agent for treating a diabetic or prediabetic patient.

2. The method of claim 1, the method further comprising selecting an agent that modulates insulin sensitivity.

3. The method of claim 1, wherein step (ii) comprises selecting an agent that increases expression of the CX3CR1 polypeptide.

4. The method of claim 1, wherein step (ii) comprises selecting an agent that increases the activity of the CX3CR1 polypeptide.

5. The method of claim 1, comprising contacting a cell expressing a CX3CR1 polypeptide or ligand-binding fragment thereof.

6. The method of claim 5, comprising detecting AKT/PKB phosphorylation.

7. The method of claim 6, wherein the cell is not treated with insulin.

8. The method of claim 6, wherein the cell is treated with insulin.

9. The method of claim 5, comprising detecting kinase activity of AKT/PKB.

10. The method of claim 9, wherein the kinase activity is detected in vitro.

11. The method of claim 5, comprising detecting p42/p44 MAP kinase phosphorylation or activity.

12. The method of claim 11, wherein the cell is not treated with insulin.

13. The method of claim 11, wherein the cell is treated with insulin.

14. The method of claim 11, wherein the kinase activity is detected in vitro.

15. The method of claim 5, comprising detecting phosphorylation of SAPK/JNK1 or p38/SAPK2.

16. The method of claim 5, comprising detecting activity of SAPK/JNK1 or p38/SAPK2.

17. The method of claim 16, wherein the kinase activity is detected in vitro.

18. The method of claim 5, comprising detecting calcium flux in a cell.

19. The method of claim 1, wherein the contacting step is performed in vitro.

20. The method of claim 1, wherein the CX3CR1 polypeptide or ligand-binding fragment thereof is expressed in a cell and the cell is contacted with the agent.

21. The method of claim 1, comprising administering the agent to an animal having diabetes and the testing the animal for decreased blood glucose levels compared to blood glucose levels before administration of the agent.

22. The method of claim 1, comprising administering the agent to an animal exhibiting insulin resistance and testing the animal for decreased insulin levels compared to insulin levels before administration of the agent.

23. The method of claim 1, further comprising the steps of contacting a cell expressing a CX3CR1 polypeptide or ligand-binding fragment thereof with the agent and testing the cell for modulated insulin sensitivity.

24. The method of claim 23, wherein insulin sensitivity is measured as a function of GLUT4 translocation or glucoase uptake in the cell.

25. The method of claim 1, wherein the amino acid sequence comprises SEQ ID NO:8.

26. The method of claim 1, wherein the amino acid sequence comprises SEQ ID NO:10.

27. The method of claim 1, wherein the amino acid sequence comprises SEQ ID NO:12.

28. A method of treating a prediabetic or diabetic animal, the method comprising administering a therapeutically effective amount of an agent that increases CX3CR1 activity or expression.

29. The method of claim 28, wherein the agent is identified in a method comprising the steps of:
(i) contacting a solution comprising an CX3CR1 polypeptide or ligand-binding fragment thereof with the agent, wherein the CX3CR1 polypeptide or ligand-binding fragment thereof is encoded by a nucleic acid that hybridizes under stringent conditions to a nucleic acid encoding a polypeptide having an amino acid sequence selected from the group consisting of SEQ ID NO:8, SEQ ID NO:10 and SEQ ID NO:12; and
(ii) selecting an agent that increases the expression or activity of the CX3CR1 polypeptide or ligand-binding fragment thereof, thereby identifying an agent for treating a diabetic or prediabetic patient.

30. The method of claim 28, wherein the animal is a human.

31. The method of claim 28, wherein animal is prediabetic.

32. The method of claim 28, wherein the animal is diabetic.

33. A method of treating a pre-diabetic or diabetic animal, the method comprising
administering a therapeutically effective amount of a polypeptide comprising an amino acid sequence at least 70% identical to SEQ ID NO:13.

34. The method of claim 33, wherein the animal is a human.

35. The method of claim 33, wherein the amino acid sequence comprises SEQ ID NO:13.

36. The method of claim 33, wherein the polypeptide sequence comprises SEQ ID NO:2.

37. A method of introducing an expression cassette into a cell, the method comprising,
introducing into the cell an expression cassette comprising a promoter operably, linked to a polynucleotide encoding a polypeptide comprising an amino acid sequence at least 70% identical to SEQ ID NO:13.

38. The method of claim 37, wherein the polypeptide comprises SEQ ID NO:13.

39. The method of claim 37, wherein the polypeptide comprises SEQ ID NO:2.

40. The method of claim 37, wherein the cell is is selected from the group consisting of adipocytes, skeletal muscle, liver and blood cells.

41. The method of claim 37, wherein the cell is introduced into a patient.

42. The method of claim 41, wherein the patient is diabetic.

43. The method of claim 41, wherein the patient is prediabetic.

44. The method of claim 41, wherein the cell is from the patient.

45. The method of claim 37, wherein the expression cassette is introduced into the cell in a viral vector.

46. A method of diagnosing individuals who have Type 2 diabetes or are prediabetic, the method comprising,
detecting in a sample from the individual the level of a CX3CR1 polypeptide or the level of a polynucleotide encoding a CX3CR1 polypeptide, wherein the CX3CR1 polypeptide is encoded by a nucleic acid that hybridizes under stringent conditions to a nucleic acid encoding a polypeptide having an amino acid sequence selected from the group consisting of SEQ ID NO:8, SEQ ID NO:10, and SEQ ID NO:12,
wherein an increased level of the polypeptide or polynucleotide in the sample compared to a level of the polypeptide or polynucleotide in either a lean individual or a previous sample from the individual indicates that the individual is diabetic or prediabetic.

47. The method of claim 46, wherein the detecting step comprises contacting the sample with an antibody that specifically binds to the CX3CR1 polypeptide.

48. The method of claim 46, wherein the amino acid sequence comprises SEQ ID NO:8.

49. The method of claim 46, wherein the amino acid sequence comprises SEQ ID NO:10.

50. The method of claim 46, wherein the amino acid sequence comprises SEQ ID NO:12.

51. The method of claim 46, wherein the detecting step comprises quantifying mRNA encoding the CX3CR1 polypeptide.

52. The method of claim 51, wherein the mRNA is reverse transcribed and amplified in a polymerase chain reaction.

53. The method of claim 46, wherein the sample is a tissue sample.

54. A method of diagnosing Type 2 diabetes or a predisposition for Type 2 diabetes in a patient, the method comprising,
detecting in a biological sample from the patient the level of fractalkine,
wherein an increased level of the fractalkine in the sample compared to a level of fractalkine in either a non-diabetic individual or a previous sample from the patient indicates that the patient is diabetic or prediabetic; and
wherein the fractalkine polypeptide is encoded by a nucleic acid that hybridizes under stringent conditions to a nucleic acid encoding a polypeptide having an amino acid sequence selected from the group consisting of SEQ ID NO:2, SEQ ID NO:4, SEQ ID NO:6 and SEQ ID NO:13.

55. The method of claim 54, wherein the detecting step comprises contacting the sample with an antibody that specifically binds to fractalkine.

56. The method of claim 54, wherein the sample is selected from the group consisting of a tissue sample, blood sample, saliva sample, and urine sample.

57. The method of claim 54, wherein the polypeptide comprises SEQ ID NO:13.

58. The method of claim 54, wherein the polypeptide comprises SEQ ID NO:2.


SEQUENCE LISTING (25 sequences)

SEQ ID NO: 1 - DNA, 3310 bp, Homo sapiens
  chemokine (C-X3-C motif) ligand 1 (CX3CL1), small inducible cytokine subfamily D (Cys-X3-Cys), member 1, SCYD1, fractalkine, neurotactin cDNA
ggcacgagggcactgagctctgccgcctggctctagccgcctgcctggcccccgccgggactcttgcccaccctcagccatggctccgatatctctgtcgtggctgctccgcttggccaccttctgccatctgactgtcctgctggctggacagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtgtgaactcctctggcctgtgtctagttgtttgattcagacagctgcctgggatccctcatcctcatacccacccccacccaagggcctggcctgagctgggatgattggaggggggaggtgggatcctccaggtgcacaagctccaagctcccaggcattccccaggaggccagccttgaccattctccaccttccagggacagagggggtggcctcccaactcaccccagccccaaaactctcctctgctgctggctggttagaggttccctttgacgccatcccagccccaatgaacaattatttattaaatgcccagccccttctgacccatgctgccctgtgagtactacagtcctcccatctcacacatgagcatcaggccaggccctctgcccactccctgcaacctgattgtgtctcttggtcctgctgcagttgccagtcaccccggccacctgcggtgctatctcccccagccccatcctctgtacagagcccacgcccccactggtgacatgtcttttcttgcatgaggctagtgtggtgtttcctgggcactgcttccagtgaggctctgcccttggttaggsattgtgggaaggggagataagggtatctggtgactttcctctttggtctacactgtgctgagtctgaaggctgggttctgatcctagttccaccatcaagccaccaacatactcccatctgtgaaaggaaagagggaggtaaggaatacctgtccccctgacaacactcattgacctgaggcccttctctccagcccctggatgcagcctcacagtccttaccagcagagcaccttagacagtccctgccaatggactaacttgtctttggaccctgaggcccagagggcctgcargggagtgagttgatagcacagaccctgccctgtgggcccccaaatggaaatgggcagagcagagaccatccctgaaggccccgcccaggcttagtcactgagacagcccgggctctgcttcccatcacccgctaagagggagggagggctccagacacatgtccaagaagcccaggaaaggctccaggagcagccacattcctgatgcttcttcagagactcctgcaggcagccaggccacaagacccttgtggtcccaccccacacacgccagattctttcctgaggctgggctcccttcccacctctctcactccttgaaaacactgttctctgccctccaagaccttctccttcacctttgtccccaccgcagacaggaccaggggatttccatgatgttttccatgagtcccctgtttgtttctgaaagggacgctacccgggaagggggctgggacatgggaaaggggaagttgtaggcataaagtcaggggttcccttttttggctgctgaaggctcgagcatgcctggatggggctgcaccggctggcctggcccctcagggtccctggtggcagctcacctctcccttggattgtccccgacccttgccgtctacctgaggggcctcttatgggctgggttctacccaggtgctaggaacactccttcacagatgggtgcttggaggaaggaaacccagctctggtccatagagagcaaaacgctgtgctgccctgcccaccctggcctctgcactcccctgctgggtgtggcgcagcatattcaggaagctcagggccctggctcaggtggggtcactctggcagctcagagagggtgggagtgggtccaatgcactttgttctggctcttccaggctgggagagcctttcaggggtgggacaccctgtgatggggccctgcctcctttgtgaggaagccgctggggccagttggtcccccttccatggactttgttagtttctccaagcaggacatggacaaggatgatctaggaagactttggaaagagtaggaagactttggaaagacttttccaaccctcatcaccaacgtctgtgccattttgtattttactaataaaatttaaaagtcttgtgaaaaaaaaaaaaaaaaaaaaaaa

SEQ ID NO: 2 - protein, 397 aa, Homo sapiens
  chemokine (C-X3-C motif) ligand 1 (CX3CL1), small inducible cytokine subfamily D (Cys-X3-Cys), member 1, SCYD1, fractalkine, neurotactin
MAPISLSWLLRLATFCHLTVLLAGQHHGVTKCNITCSKMTSKIPVALLIHYQQNQASCGKRAIILETRQHRLFCADPKEQWVKDAMQHLDRQAAALTRNGGTFEKQIGEVKPRTTPAAGGMDESVVLEPEATGESSSLEPTPSSQEAQRALGTSPELPTGVTGSSGTRLPPTPKAQDGGPVGTELFRVPPVSTAATWQSSAPHQPGPSLWAEAKTSEAPSTQDPSTQASTASSPAPEENAPSEGQRVWGQGQSPRPENSLEREEMGPVPAHTDAFQDWGPGSMAHVSVVPVSSEGTPSREPVASGSWTPKAEEPIHATMDPQRLGVLITPVPDAQAATRRQAVGLLAFLGLLFCLGVAMFTYQSLQGCPRKMAGEMAEGLRYIPRSCGSNSYVLVPV

SEQ ID NO: 3 - DNA, 3155 bp, Mus musculus
  fractalkine/neurotactin, CX3C chemokine cDNA
cgggaaacagctatgacccaagcatacaatcaactccaagctcggaattttaattaagaattcctgcactccagccatggctccctcgccgctcgcgtggctgctgcgcctggccgcgttcttccatttgtgtactctgctgccgggtcagcacctcggcatgacgaaatgcgaaatcatgtgcgacaagatgacctcacgaatcccagtggctttgctcatccgctatcagctaaaccaggagtcctgcggcaagcgtgccattgtcctggagacgacacagcacagacgcttctgtgctgacccgaaggagaaatgggtccaagacgccatgaagcatctggatcaccaggctgctgccctcactaaaaatggtggcaagtttgagaagcgggtggacaatgtgacacctgggatcaccttggccactaggggactgtccccatctgccctgacaaagcctgaatccgccacattggaagaccttgctttggaactgactactatttcccaggaggccagggggaccatggggacttcccaagagccaccggcagcagtgaccggatcatctctctcaacttccgaggcacaggatgcagggcttacggctaagcctcagagcattggaagttttgaggcggctgacatctccaccaccgtttggccgagtcctgctgtctaccaatctggatctagctcctgggctgaggaaaaagctactgagtccccctccactacagccccatctcctcaggtgtccactacttcaccttcaaccccagaggaaaatgttgggtccgaaggccaacccccatgggtccagggacaggacctcagtccagagaagtctctagggtctgaggagataaacccagttcatactgataatttccaggagagggggcctggcaacacagtccacccctcagtggctcccatctcctctgaagagacccccagcccagagctggtggcctcgggcagccaggctcctaagatagaggaacccatccatgccactgcagatccccagaaactgagtgtgcttatcactcctgtccccgacacccaggcagccacaaggaggcaggcagtggggctactggctttccttggtcttcttttctgcctaggggtggccatgtttgcttaccagagccttcagggctgtccccgcaaaatggcgggggaaatggtagaaggcctccgctacgtcccccgtagctgtggcagtaactcatacgtcctggtgccagtgtgagctgcttgcctgcctgcctgtgtccagagtgtgattcggacagctgtctggggacccccccccatcctcatacccaccttcatccacgctggggaaatgggaatggagaagctggaccctccaggggctgtgggctccatccaatcccccctcccccgaggggtggccccggaggccaccctagaccactattcacttatcagagacagagcaggtgaccttccagctcctctatatttgaaagaatcctctgctgctggctggttagaggggccttgacaccccaactccagtgaacaattatttattggattcccagcccctgcgacgacacctgtttcccgcgcgcaccgtggtccgcccatatcacaagcagcaggccaggcctatctgcctgtccccctgacctccttgtgtctcctggctttgctgcagtcgccagcccttctcctccccggccagctgcggtgctatctgccctatgtctccctctatcccctgtacagagcgcaccaccatcaccatcaacaccgctgttgtgtcttttcttgcatgaggttaaagctgtgttttctggagctctccgggaagggagacaagcttgcgagagggtttaaagtgttcctccccagacttggatgtgctgtgaaggcatgctgcgtctgaaggaagggtccagtccccactcggctaccagcaccacaaagtgccccacctgtaaaaggaaagaaacgtggtccagagctggcaataacctatggccctgacatcatcactttctctgagatccttgtctccacccctgggtgcaaccccaccccttatcaacattaatagtcactgccattccactggactgacatttttgtaccctgtgattctgagggctggcaaggagtggcttgagagtgcagatcgtaccctgtatgcccccccccaaatggaggctgagttggggacttgcaggaacagaggccaactcagatggcttcccctgtgttctcactagaaacccctcccccatgcaccaaggtgacagtcacaggtctgccctggctaaaggacaagccacataggaaagattaggacaagcccctcggaggcagaggatccagggtaaacccctggagtggccacaaacccaatttcagtgtagggacttgtgcatgtgtgtacttgcatagtcagacagaggctgccagggtcctttcctgtctctgagagcagtgttcacgccaaggactcacctttgcccccattgcaggcagggccagaactcccatagcattctccaagagccctgtgacattttctggaagggactctgccctgggcgcaaagtgactgctgaagcaaggagcagctgagcagcaccccagcggagctgagccggcaggccacgcccctcgggggggggcatttctacccaccctgctctgaatagctccaacttcaccttaggagcctcccaggggcgagcttcacccagaagccagtgactcactccttgattggtggaagctcagttggctcctgagagtgaggaagccaaccctttgtcgaccctcctcctgggaagcctgtgggcggctctgatcatgctccacagaaccagttgtaggcctgagccgcagcagcccgagtgcactatatcctggctccttcggtggggaacctttaagggttgggacacccgtcatcggactttgttggttcctccctcccagagcagaatgtgggccgtaacaatctgaggaggactttgaaagttgttgatcctttagggttttttttcaagcatcattaccaatgtctgtgccattttgtattttactaataaaattttaaagtcttgtgaattaaaaaaaaaaaaaaaaaaaaaaaaaaaaaa

SEQ ID NO: 4 - protein, 395 aa, Mus musculus
  fractalkine/neurotactin, CX3C chemokine
MAPSPLAWLLRLAAFFHLCTLLPGQHLGMTKCEIMCDKMTSRIPVALLIRYQLNQESCGKRAIVLETTQHRRFCADPKEKWVQDAMKHLDHQAAALTKNGGKFEKRVDNVTPGITLATRGLSPSALTKPESATLEDLALELTTISQEARGTMGTSQEPPAAVTGSSLSTSEAQDAGLTAKPQSIGSFEAADISTTVWPSPAVYQSGSSSWAEEKATESPSTTAPSPQVSTTSPSTPEENVGSEGQPPWVQGQDLSPEKSLGSEEINPVHTDNFQERGPGNTVHPSVAPISSEETPSPELVASGSQAPKIEEPIHATADPQKLSVLITPVPDTQAATRRQAVGLLAFLGLLFCLGVAMFAYQSLQGCPRKMAGEMVEGLRYVPRSCGSNSYVLVPV

SEQ ID NO: 5 - DNA, 3044 bp, Rattus norvegicus
  chemokine CX3C, fractalkine cDNA
tgcactccagccatccagccatggctccctcacagctcgcgtggctgctgcgcctggccgcgttctttcatctgtgtactctgctggcgggtcagcacctcggcatgacgaaatgcaacatcacgtgccacaagatgacctcgccaatcccagtgaccttgctcatccactatcaactgaaccaggagtcctgcggcaagcgcgccatcatcctggagacgagacagcacagacacttctgtgctgacccaaaggagaaatgggtccaagacgccatgaagcacctggaccaccagaccgctgccctgactagaaatggtggcaagtttgagaagcgtgtggacaatgtgacacctaggatcacctcggccaccaggggactgtccccaactgccctggcaaagcctgagtcagccacagtggaagaccttactttggaaccgactgctatttcccaggaggcccgaaggcccatggggacttcccaagagccaccagcagcagtgactggatcgtctccctcaacttccaaagctcaggatgcagggcttgccgccaagcctcagagcactggaatttctgaggtggctgctgtctctaccaccatttggccgagttctgctgtctaccaatctggatctagcctctgggccgaggaaaaagctactgaatccccccccactatagccctatctactcaagcgtccaccacttcaagccccaagcagaatgttgggtctgaaggccagcccccatgggtccaggagcaggactccactccagagaaatctccagggcctgaggagacaaacccagttcatactgatattttccaagacagaggacctggcagcacagtccatccctcagtggctcccacctcctctgaaaagacccccagcccggagctggtggcctcgggcagccaggctcctaaggtagaggaacccattcatgccactgcagatccccagaaactgagtgtgtttatcactcctgtccctgactcccaggcagccacccggaggcaggcagtggggttactggcctttcttggtctactcttctgtctgggggtggccatgtttgcctaccagagcctccagggctgtccccgcaaaatggcaggggagatggtggaaggcctccgctacgtcccccgtagctgtggcagtaactcgtacgtcctggtgccggtgtgagctgcctgcttgcctgcctgtgtccagagtgtgattcagacagctgtctggggacccccatcctcatacccaccttcatccacgctgggagaatgggaatgcagaagctagatcctccaggggccatgtgctccaacccccgaggagtggccctggaggccaccctagaccattattcacttatcagagacagacagagcaggtggccttccagctcccccatatttgaaagaatcctctgctgctggctggttagaggggcccttgacatcccaaactctatgagcaattatttattggattcccagccccctgagacacccgtttccccgtgcgcaccgtggtccacccatcttacaagcagcagccaggcctctctgcctgtcccctgacctcctcgtgtctcctggctttgctgcagtcgccagccctgtctcctccccggccagctgcggtgctatctatcccggtctctctctctcgtgtacagagccatcaccaccatcaccaccaacaactcgttctgtctttgcttgcatgaggttaatgctgtgttttctggagctctctgggacgggagatgagcttctgtgagggtttaaagtgttcctccccagactttgatgtgccgtgaagcatgctgcctctgaaggaaggctctggtccccactccgctgccagcacaacaaagtgccccacctgtaaaggaaaagagactcggcccagagctggcaaaaacccatggccctgacatcatcactttctctgagatctttgtccccatccctggatgccaccccccccccccagcccttatcaacatgaatagtcactgccattccactggactgacacttttgtatgctgtgattctgagggctggcaagggatgacttgagagtgcagatcctacccatgggcccccaaatggaggctgagctggggacctgcaggaagagaggccaactcagaaggctccgctgtgttctcactggcacccctcccctgtgcaccaaggtgacagtcacaggtctgccctgcctgaagaacaagccacagaaggaagattatgacaagtccctgggaggccaaggatccagggcaaaccctggagtggccacacacccaatttcagcttagggacttgtgcatgtgtgtacttgcacagcccagatcattcagaagctgccaggatcctttcctacatctgagagcgcagttcctgccaaggtctcaccttcgcctcacttcaggcagggcagaactcccataacattctccaagagccctgtgacgtgttctggaagggactctgccctgggcacaaagtgtctactgaagcagagagcagcgccccagccccagccccagcgccccagcgggagctgtaccggcagaccacgccctgggggggagggccctccgtggcagctttccgctctgaatagctccaaccgtcacctttggagcctcccaggggcgggcttcacccagccagtgactcactccttgataggtggaagctcagaacaggtggtctcgtcccagagtgaggaagccagccccttggcgaccctcctcttgggaagcttgtgggaggctctggtctggctccagagtactagttgtaggcctgaggagcagcagcccgagtgcactatatcctggttcttcggtggggagccttcaagggttgggacacccatggttggactttgttggttcccgcgcctcgtgggccaaaacaatctgaggaggactttggaaggagttggtttttcaagcatcattaccaatgtctgtgccattttgtattttactaataaaattttaaagtcttgt

SEQ ID NO: 6 - protein, 393 aa, Rattus norvegicus
  chemokine CX3C, fractalkine
MAPSQLAWLLRLAAFFHLCTLLAGQHLGMTKCNITCHKMTSPIPVTLLIHYQLNQESCGKRAIILETRQHRHFCADPKEKWVQDAMKHLDHQTAALTRNGGKFEKRVDNVTPRITSATRGLSPTALAKPESATVEDLTLEPTAISQEARRPMGTSQEPPAAVTGSSPSTSKAQDAGLAAKPQSTGISEVAAVSTTIWPSSAVYQSGSSLWAEEKATESPPTIALSTQASTTSSPKQNVGSEGQPPWVQEQDSTPEKSPGPEETNPVHTDIFQDRGPGSTVHPSVAPTSSEKTPSPELVASGSQAPKVEEPIHATADPQKLSVFITPVPDSQAATRRQAVGLLAFLGLLFCLGVAMFAYQSLQGCPRKMAGEMVEGLRYVPRSCGSNSYVLVPV

SEQ ID NO: 7 - DNA, 3100 bp, Homo sapiens
  chemokine (C-X3-C) receptor 1 (CX3CR1), G protein-coupled receptor V28 cDNA
actcgtctctggtaaagtctgagcaggacagggtggctgactggcagatccagaggttcccttggcagtccacgccaggccttcaccatggatcagttccctgaatcagtgacagaaaactttgagtacgatgatttggctgaggcctgttatattggggacatcgtggtctttgggactgtgttcctgtccatattctactccgtcatctttgccattggcctggtgggaaatttgttggtagtgtttgccctcaccaacagcaagaagcccaagagtgtcaccgacatttacctcctgaacctggccttgtctgatctgctgtttgtagccactttgcccttctggactcactatttgataaatgaaaagggcctccacaatgccatgtgcaaattcactaccgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcctccaggaaatctggcccgtgctccgcaatgtggaaacaaattttcttggcttcctactccccctgctcattatgagttattgctacttcagaatcatccagacgctgttttcctgcaagaaccacaagaaagccaaagccattaaactgatccttctggtggtcatcgtgtttttcctcttctggacaccctacaacgttatgattttcctggagacgcttaagctctatgacttctttcccagttgtgacatgaggaaggatctgaggctggccctcagtgtgactgagacggttgcatttagccattgttgcctgaatcctctcatctatgcatttgctggggagaagttcagaagatacctttaccacctgtatgggaaatgcctggctgtcctgtgtgggcgctcagtccacgttgatttctcctcatctgaatcacaaaggagcaggcatggaagtgttctgagcagcaattttacttaccacacgagtgatggagatgcattgctccttctctgaagggaatcccaaagccttgtgtctacagagaacctggagttcctgaacctgatgctgactagtgaggaaagatttttgttgttatttcttacaggcacaaaatgatggacccaatgcacacaaaacaaccctagagtgttgttgagaattgtgctcaaaatttgaagaatgaacaaattgaactctttgaatgacaaagagtagacatttctcttactgcaaatgtcatcagaactttttggtttgcagatgacaaaaattcaactcagactagtttagttaaatgagggtggtgaatattgttcatattgtggcacaagcaaaagggtgtctgagccctcaaagtgaggggaaaccagggcctgagccaagctagaattccctctctctgactctcaaatcttttagtcattatagatcccccagactttacatgacacagctttatcaccagagagggactgacacccatgtttctctggccccaagggaaaattcccagggaagtgctctgataggccaagtttgtatcaggtgcccatccctggaaggtgctgttatccatggggaagggatatataagatggaagcttccagtccaatctcatggagaagcagaaatacatatttccaagaagttggatgggtgggtactattctgattacacaaaacaaatgccacacatcacccttaccatgtgcctgatccagcctctcccctgattacaccagcctcgtcttcattaagccctcttccatcatgtccccaaacctgcaagggctccccactgcctactgcatcgagtcaaaactcaaatgcttggcttctcatacgtccaccatggggtcctaccaatagattccccattgcctcctccttcccaaaggactccacccatcctatcagcctgtctcttccatatgacctcatgcatctccacctgctcccaggccagtaagggaaatagaaaaaccctgcccccaaataagaagggatggattccaaccccaactccagtagcttgggacaaatcaagcttcagtttcctggtctgtagaagagggataaggtacctttcacatagagatcatcctttccagcatgaggaactagccaccaactcttgcaggtctcaacccttttgtctgcctcttagacttctgctttccacacctgcactgctgtgctgtgcccaagttgtggtgctgacaaagcttggaagagcctgcaggtgccttggccgcgtgcatagcccagacacagaagaggctggttcttacgatggcacccagtgagcactcccaagtctacagagtgatagccttccgtaacccaactctcctggactgccttgaatatcccctcccagtcaccttgtgcaagcccctgcccatctgggaaaataccccatcattcatgctactgccaacctggggagccagggctatgggagcagcttttttttcccccctagaaacgtttggaacaatgtaaaactttaaagctcgaaaacaattgtaataatgctaaagaaaaagtcatccaatctaaccacatcaatattgtcattcctgtattcacccgtccagaccttgttcacactctcacatgtttagagttgcaatcgtaatgtacagatggttttataatctgatttgttttcctcttaacgttagaccacaaatagtgctcgctttctatgtagtttggtaattatcattttagaagactctaccagactgtgtattcattgaagtcagatgtggtaactgttaaattgctgtgtatctgatagctctttggcagtctatatgtttgtataatgaatgagagaataagtcatgttccttcaagatcatgtaccccaatttacttgccattactcaattgataaacatttaacttgtttccaatgtttagcaaatacatattttatagaacttc

SEQ ID NO: 8 - protein, 355 aa, Homo sapiens
  chemokine (C-X3-C) receptor 1 (CX3CR1), G protein-coupled receptor V28
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 9 - DNA, 2813 bp, Mus musculus
  chemokine receptor CX3CR1
aattgttcaccctttcagtgttttctcccgcttgctgcatgcagccagtgagaaccgcgatcctctaagactcacgtgatctggtttgctgcatacagccagtgagaactgcgatcctctaagactcacgtggacctgcttactgcatgcagccagtgagaactacaatcctttaaggctcacgtgatctggtttctccttcccctccaggacctcaccatgtccacctccttccctgaactggatctagagaattttgagtatgacgattctgctgaggcctgttatttgggcgacattgtggcctttggaaccatcttcctgtccgtcttctacgccctcgtcttcacgttcggtctggtgggaaatctgttggtggtcctcgctctcaccaacagccggaagcccaagagcatcactgacatctacctcctgaacctggccttgagcgacctgctctttgtggccaccttgcccttctggactcactacctcatcagccatgagggcctccacaatgccatgtgcaagctcacgactgccttcttcttcattggcttctttgggggcatattcttcatcaccgtcatcagcatcgaccggtaccttgccatcgtcctggccgccaactccatgaacaaccggacagtgcagcacggtgtcaccattagtctgggcgtctgggcggcggccatcttagtggcgtcaccccagttcatgttcacaaagagaaaggacaacgagtgtctgggtgactaccccgaggtcctgcaggaaatgtggcccgtgctccgcaactcggaagtcaacatcctgggcttcgccctgcccttgcttatcatgagcttttgctacttccgcatcatccagacgctgttttcctgcaagaatcgcaagaaggccagagccgtcagactcatcctcctggtggtctttgccttcttcctcttctggacaccatacaacatcatgattttcctggagactctcaagttctacaacttcttccccagttgtgacatgaagagggacctgaggttggccctcagtgtgacggagacagtggcgttcagccactgttgcctcaacccctttatctacgcctttgccggggaaaagttcagaagatacctgggacacctgtataggaagtgcctggccgtcctgtgcggtcatcctgtccacaccggcttctcgccagagtcccagaggagcaggcaggacagcattctgagcagtttcactcactacacaagcgagggagatgggtctctcctgctctgaaggggtctccccgaccctagctccactaggaacccagagttcttgcatcagatttccctgccgctccccctgcatcttatgtgcaagaaatatggaccagatgcctgcaaaccaaccccgtggtgtttttttgaaaaatttatgttcaatgtgtgaaaaacacacgtatctcttactgcaaatgttgaacattggggcttactggtgacaaaaattctaaccagattagtgcaattacaaaggggtttggtgagtcctggttgcatgatcatgtgataaaggacaactaagtcctcagactgagtggaaaccaaggcttggctccaatgtcccctctctgaccttcagatccttcatagtgacagatcattccaggttctatcatcagagaaggaccacatctctctgatttcaaaattggtattcctagggaacacctccgttggccgagtgtgtcgggtgtccattccttgactaggtggtgttattgaaatagaagggatacctaagatgctgttggaatctcaaggttagcggttgagagagacacatctctcagaagctggggggtgggagctactctgacagcaagaactgctgactcgccttaccatggagctcattcaggctccccttcagtaactagcagtatctgttgctagcttctttaatcttctgttgagaatgtcctgaactctccaagggttagaatttgggttactgctcacagcatcaaattcaatcccaaggccctgtcctccaagaccaggaagataggatgcagttctaacaagagactccacgctgactcctcattccacaggactccgtccacccagttggccatgtccctttttcttgtcttgacccacatccctccactcttctcagcccaggggaagaaatagaaagagccatgccccagagtgaagggatcgattcatatcccaactctgttgcttgcacattggggagactggagccaacagagcttcagtcccttcaatatagaaaggggactgtggtgccttcatccattctagcatagtacctcagcaaggactttagcattccctggatcaggaactgttaccctgcctctgagaaatggagctggtggagtgggaggaacctgcaggttgctgcaggacacagccagacaagaggagacctattcttacagcatcccccaacatgtgcctagatctacaaagccattcccatgtccccgctgtcagggcaagacccaggccacgagagcagcttgctttctcccctgaccctgcaagcatcacgtaggatgtagccacaagaatgtcttcatgcattcactgactccggtctcatttgcaggcttacattgaaagttccagttctgcaacttgctttttaaaaaacactggatttcaggggctggagagatggctcagtggttaagagcacagtctgctcctacagaggtcccaagttcaattcccatgtcatgtctgaagacaaggacagtgtagtccaaaaaaaaactggaattc

SEQ ID NO: 10 - protein, 354 aa, Mus musculus
  chemokine receptor CX3CR1
MSTSFPELDLENFEYDDSAEACYLGDIVAFGTIFLSVFYALVFTFGLVGNLLVVLALTNSRKPKSITDIYLLNLALSDLLFVATLPFWTHYLISHEGLHNAMCKLTTAFFFIGFFGGIFFITVISIDRYLAIVLAANSMNNRTVQHGVTISLGVWAAAILVASPQFMFTKRKDNECLGDYPEVLQEMWPVLRNSEVNILGFALPLLIMSFCYFRIIQTLFSCKNRKKARAVRLILLVVFAFFLFWTPYNIMIFLETLKFYNFFPSCDMKRDLRLALSVTETVAFSHCCLNPFIYAFAGEKFRRYLGHLYRKCLAVLCGHPVHTGFSPESQRSRQDSILSSFTHYTSEGDGSLLL

SEQ ID NO: 11 - DNA, 1318 bp, Rattus norvegicus
  putative G-protein coupled receptor (GCR) cDNA
gtgggactgggtgagtggctggcacttcctgcagaagtccccgtccccagctgctcaggacctcaccatgcctacctccttcccggaattggatctagagaactttgagtatgatgactctgctgaggcctgttatttgggtgacatcgtggcctttgggaccatcttcctatctattttctactcccttgtcttcacgttcggtctggtggggaatctgttggtggtcctcgccctcaccaacagccggaagtccaagagcatcactgacatctacctcctgaacctggccttgagcgacctgctctttgtggccactttgcccttctggactcactacctcatcagccatgagggcctccacaacgccatgtgcaagctcacgactgctttcttcttcattggcttctttgggggcatattcttcatcaccgtcatcagcatcgaccggtacctcgccatcgtcctggccgccaactccatgaacaaccggacagtgcaacacggcgtcaccatcagtctgggcgtctgggcggcggccatcttagtggcgtcgccccagttcatgttcacaaagagaaaggacaacgaatgtttgggtgattaccccgaggtcctgcaggaaatctggcccgtgctccgcaactcggaggtcaacatcctgggcttcgtcctgcccttgcttatcatgagcttttgctacttccgcatcgtccggacgctgttttcctgcaagaaccggaagaaggccagagccattaggctcatcctcttggtggttgttgtcttcttcctcttctggacgccttacaacatcgtgattttcctggagactctcaaattctacaacttcttccctagttgtggcatgaagagggacctgaggtgggcccttagtgtgacggagacagtggcgtttagccactgctgcctcaacccctttatctacgctttcgctggggaaaagttcagaaggtacctgagacacctgtacaacaagtgcctggccgtcctgtgcggtcgtcctgtccacgccggcttctcaacagagtcccagaggagcaggcaggacagcattctgagcagcttgactcactacacaagcgagggagagggatctctcctgctctgaagggtctccccgaccccgactctactaagaacccagagttcctgcatctgactctgtgtaatgaaaacagattcacacacacacacacacacacacacacacacacacacacacacaccccgctcctcctgcattttatgtgcaagaaatacggaccaggtacctgcaaatcaatccacagtgttt

SEQ ID NO: 12 - protein, 354 aa, Rattus norvegicus
  putative G-protein coupled receptor (GCR)
MPTSFPELDLENFEYDDSAEACYLGDIVAFGTIFLSIFYSLVFTFGLVGNLLVVLALTNSRKSKSITDIYLLNLALSDLLFVATLPFWTHYLISHEGLHNAMCKLTTAFFFIGFFGGIFFITVISIDRYLAIVLAANSMNNRTVQHGVTISLGVWAAAILVASPQFMFTKRKDNECLGDYPEVLQEIWPVLRNSEVNILGFVLPLLIMSFCYFRIVRTLFSCKNRKKARAIRLILLVVVVFFLFWTPYNIVIFLETLKFYNFFPSCGMKRDLRWALSVTETVAFSHCCLNPFIYAFAGEKFRRYLRHLYNKCLAVLCGRPVHAGFSTESQRSRQDSILSSLTHYTSEGEGSLLL

SEQ ID NO: 13 - protein, 76 aa, Artificial Sequence
  Description of Artificial Sequencehuman fractalkine chemokine domain
QHHGVTKCNITCSKMTSKIPVALLIHYQQNQASCGKRAIILETRQHRLFCADPKEQWVKDAMQHLDRQAAALTRNG

SEQ ID NO: 14 - DNA, 228 bp, Artificial Sequence
  Description of Artificial Sequencehuman fractalkine chemokine domain
cagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggc

SEQ ID NO: 15 - protein, 200 aa, Artificial Sequence
  Description of Artificial Sequencepoly-Gly flexible linker
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

SEQ ID NO: 16 - DNA, 23 bp, Artificial Sequence
  Description of Artificial SequenceCX3CR1 6-fam Taqman probe
aatgcctggctgtcctgtgtggg

SEQ ID NO: 17 - DNA, 27 bp, Artificial Sequence
  Description of Artificial SequenceCX3CR1 forward PCR primer
cagaagatacctttaccacctgtatgg

SEQ ID NO: 18 - DNA, 23 bp, Artificial Sequence
  Description of Artificial SequenceCX3CR1 reverse PCR primer
gaggagaaatcaacgtggactga

SEQ ID NO: 19 - DNA, 25 bp, Artificial Sequence
  Description of Artificial SequenceFractalkine 6-fam Taqman probe
caaacgcgcaatcatcttggagacg

SEQ ID NO: 20 - DNA, 21 bp, Artificial Sequence
  Description of Artificial SequenceFractalkine forward PCR primer
tcaacagaaccaggcatcatg

SEQ ID NO: 21 - DNA, 18 bp, Artificial Sequence
  Description of Artificial SequenceFractalkine reverse PCR primer
cgggtcggcacagaacag

SEQ ID NO: 22 - DNA, 25 bp, Artificial Sequence
  Description of Artificial SequenceCX3CR1 forward PCR primer
agaagggatacctaagatgctgttg

SEQ ID NO: 23 - DNA, 17 bp, Artificial Sequence
  Description of Artificial SequenceCX3CR1 reverse PCR primer
ccaccccccagcttctg

SEQ ID NO: 24 - DNA, 27 bp, Artificial Sequence
  Description of Artificial SequenceNeurotactin forward PCR primer
ccaactccagtgaacaattatttattg

SEQ ID NO: 25 - DNA, 15 bp, Artificial Sequence
  Description of Artificial SequenceNeurotactin reverse PCR primer
gcgcgcgggaaacag